(19) United States
(12) Patent Application Publication
(10) Pub. No.: US 2021/0137850 A1
(43) Pub. Date: May 13, 2021

(54) DIAGNOSIS & TREATMENT OF ERCC3-MUTANT CANCER

(71) Applicant: MEMORIAL SLOAN KETTERING CANCER CENTER, New York, NY (US)

(72) Inventors: Vijai JOSEPH, New York, NY (US); Kenneth OFFIT, New York, NY (US); Sabine TOPKA, New York, NY (US)

(21) Appl. No.: 16/493,214

(22) PCT Filed: Mar. 15, 2018
(86) PCT No.: PCT/US2018/022588

§ 371 Date: Sep. 11, 2019

Related U.S. Application Data
(60) Provisional application No. 62/471,941, filed on Mar. 15, 2017.

(51) Int. Cl. A61K 31/122; A61P 35/00; C12Q 1/6886

(52) U.S. Cl. CPC A61K 31/122; A61P 35/00; C12Q 2600/118; C12Q 2600/106; C12Q 2600/156; C12Q 1/6886

(57) ABSTRACT

The present invention provides various compositions and methods useful for the diagnosis and treatment of cancer, particularly in subjects having certain mutations in the ERCC3 gene, such as the R109X mutation. In some embodiments such methods involve administration of a class of DNA alkylating agents known as Illudins to the subject.

### CROSS-REFERENCE TO RELATED APPLICATIONS

This application claims the benefit of priority of U.S. Provisional Patent Application No. 62/471,941 filed on Mar. 15, 2017, the content of which is hereby incorporated by reference in its entirety.

### STATEMENT REGARDING FEDERALLY SPONSORED RESEARCH

This invention was made with government support under grant numbers CA176785 and CA008748 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 15, 2018, is named MSKCC_021_WO1_SL.TXT and is 17,412 bytes in size.

### INCORPORATION BY REFERENCE

For the purposes of only those jurisdictions that permit incorporation by reference, the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Numbers in superscript or parentheses following text herein refer to the numbered references identified in the “Reference List” section of this patent application.

### BACKGROUND

Genetic susceptibility to breast cancer has been shown to be strongly associated with rare coding gene mutations and common non-coding genomic variants (1). Loss of function mutations in well characterized genes, such as those in BRCA1/2 and other members of the homologous recombination pathway, are routinely used for clinical risk assessment in cancer, but account for a small fraction of excess familial risk (2). Common single nucleotide polymorphisms (SNPs) are yet to demonstrate clinical utility, but over 90 SNPs account for over 37% of familial relative risk (3). An as-yet to be defined proportion of the remaining familial risk is accounted for by variants of “moderate” risk, including coding or non-coding variants in the DNA repair pathways such as nucleotide excision repair (NER), mismatch repair (MMR) and base excision repair (BER), which play an important role in checkpoint and genomic integrity maintenance (4, 5).

Thus, there is a continuing need in the art to identify additional genes and mutations that may be associated with familial breast cancer risk, and to develop new and improved methods for the diagnosis and treatment of breast cancers bearing such mutations. There is also a need in the art to identify genes and mutations that may be associated with cancer risk for other cancers, and to develop new and improved methods for the diagnosis and treatment of such other cancers.

### SUMMARY OF THE INVENTION

The present invention is based, in part, on a series of important discoveries that are described in more detail in the Examples section of this patent specification. (Certain aspects of the work described in the Examples have been published by the present inventors (38).

For example, it has now been discovered that certain mutations in the ERCC3 gene show a significant association with cancer risk, including risk of breast cancer, colorectal cancer, bladder cancer, glioma and non-small cell lung cancer (NSCLC). It has now also been shown that these ERCC3 gene mutations confer increased sensitivity of cancer cells to UV irradiation and to certain chemotherapeutic agents—both in vitro and in vivo. Building on these discoveries, and other discoveries presented herein, the present invention provides a variety of new and improved methods for the diagnosis and treatment of various cancer types and for inhibiting the proliferation of cancer cells.

In some embodiments the present invention provides methods for inhibiting the proliferation of cancer cells. In some embodiments the present invention provides methods for treating cancer in a subject. In some embodiments the present invention provides “diagnostic” methods—which can be used to determine cancer risk of a subject and/or to inform treatment strategies or treatment dosages for a subject. In other embodiments the present invention provides methods for both “diagnosing” and treating cancer in a subject.

In some embodiments the cancer is breast cancer. In some embodiments the cancer is colorectal cancer. In some embodiments the cancer is NSCLC. In some embodiments the cancer is bladder cancer. In some embodiments the cancer is a glioma. In some embodiments the cancer is selected from the group consisting of breast cancer, colorectal cancer, NSCLC, bladder cancer, and glioma. In some embodiments the cancer is selected from the group consisting of colorectal cancer, NSCLC, bladder cancer, and glioma. In some embodiments the cancer is not breast cancer.

In some embodiments the cells have, or the subject has, a truncating hypomorphic mutation in the ERCC3 gene. In some embodiments the mutation in the ERCC3 gene is one that results in truncation of the ERCC3 protein within its first putative helicase domain. In some embodiments the mutation in the ERCC3 gene is an R109X mutation.

In some embodiments the present invention provides methods of inhibiting the proliferation of cancer cells that involve contacting the cancer cells with an effective amount of an Illudin. In some embodiments the present invention provides methods of treatment of cancer that involve administering an effective amount of an Illudin to a subject.

Various different Illudin molecules can be used in carrying out the methods described herein. Illudin molecules that may be used include, but are not limited to, Illudin A, Illudin B, Illudin M, Illudin S, 6-Deoxyilludin M, dehydroilludin M, dihydroilludin M, 6-Deoxyilludin S, dehydroilludin S, dihydroilludin S and Irofulven or a derivative of Irofulven. In some embodiments the Illudin is Illudin S. In some embodiments the Illudin is Irofulven.

The “effective amount” of the Illudin molecule can be determined as described in the “Detailed Description” or “Examples” sections of this patent application, and/or using standard dose determination/escalation studies, and/or as further described herein. Importantly, it has been found that cancer cells and subjects having the ERCC3 mutations described herein are more sensitive to treatment with Illudins than those not having such mutations. This is particularly important given that Illudins, such as Irofulven, have been shown to have significant toxicity. (39, 40, 41, 42). Thus, in carrying out the methods of the present invention, an “effective amount” of a given Illudin molecule may be significantly lower than the amount of that same Illudin molecule that is, or would be, needed to inhibit the proliferation of cancer cells not having the ERCC3 mutation or to treat a cancer in a subject not having the ERCC3 mutation. By way of example, the effective amount may be about 100%, or about 90%, or about 80%, or about 70%, or about 60%, or about 50%, or about 40%, or about 30%, or about 20%, or about 10%, of the Illudin's maximum tolerated dose in a subject. Similarly, the effective amount may be about 100%, or about 90%, or about 80%, or about 70%, or about 60%, or about 50%, or about 40%, or about 30%, or about 20%, or about 10%, of the dose of the Illudin that is effective for and/or approved for and/or typically used for the treatment of subjects not having the ERCC3 mutation. For example, the Illudin Irofulven has been tested in human clinical trials and found to have a maximum tolerated dose in human subjects of 18 mg/m²/infusion, or 0.55 mg/kg/infusion, or 50 mg total per infusion (39, 40, 41, 42). In some embodiments the effective amount of Irofulven used in the methods of the present invention may be about 100%, or about 90%, or about 80%, or about 70%, or about 60%, or about 50%, or about 40%, or about 30%, or about 20%, or about 10%, of such maximum tolerated doses (i.e. of 18 mg/m²/infusion, or 0.55 mg/kg/infusion, or 50 mg total per infusion).

In those treatment methods that also involve “diagnosis,” a determination may be made regarding whether a subject has, or does not have, an ERCC3 mutation, prior to commencing treatment—for example by administration of an Illudin. In some embodiments the Illudin is administered only if such a mutation is determined to be present. In some embodiments the dose of the Illudin is reduced if such a mutation is determined to be present. For example, in some embodiments, if the ERCC3 mutation is determined to be present, the dose of the Illudin is reduced to about 90%, or about 80%, or about 70%, or about 60%, or about 50%, or about 40%, or about 30%, or about 20%, or about 10%, of the dose that is effective for and/or approved for and/or typically used for the treatment of subjects not having the ERCC3 mutation, or of the maximum tolerated dose.

One important aspect of the present invention is that it has been found that the association between having an R109X mutation in the ERCC3 gene and having certain cancers is greater in certain particular groups of patients/subjects, for example in those having Ashkenazi Jewish ancestry, in those having estrogen receptor positive (ER+) breast cancers, and/or in those having BRCA-negative breast cancers. Thus, in some embodiments the subjects treated with the methods of the present invention are of Ashkenazi Jewish ancestry. Similarly, in some embodiments the subjects treated with the methods of the present invention have an ER+ breast cancer. In some embodiments the subjects treated with the methods of the present invention have a BRCA-negative breast cancer.

As mentioned above, several of the methods described herein involve performing a “diagnostic” test to determine if a subject has an ERCC3 mutation (e.g. one of the mutations described herein, such as the R109X mutation). In some embodiments such tests are performed prior to administering an Illudin to the subject. In some embodiments such tests are performed as stand-alone diagnostic tests—i.e. without necessarily subsequently treating the subject. Thus, the present invention also provides several diagnostic tests that can be used determine if a subject has an ERCC3 mutation whether or not treatment with an Illudin is also ultimately used. For example, such diagnostic tests can be performed to determine if a subject is at risk for developing cancer, and/or to determine if a subject is a candidate for potential treatment with an Illudin. In some embodiments the cancer is breast cancer. In some embodiments the cancer is colorectal cancer. In some embodiments the cancer is NSCLC. In some embodiments the cancer is bladder cancer. In some embodiments the cancer is a glioma. In some embodiments the cancer is selected from the group consisting of breast cancer, colorectal cancer, NSCLC, bladder cancer, and glioma. In some embodiments the cancer is selected from the group consisting of colorectal cancer, NSCLC, bladder cancer, and glioma. In some embodiments the cancer is not breast cancer. In some embodiments the subject is of Ashkenazi Jewish ancestry. Some of such diagnostic tests involve determining whether an ERCC3 mutation is present in the ERCC3 gene, or of a fragment thereof, in a subject, or in a tissue sample, cell sample, or nucleic acid sample obtained from the subject. Conversely, some of such diagnostic tests involve determining whether a mutant ERCC3 protein is present in the subject, or in a tissue sample, cell sample, or protein sample obtained from the subject.

Further details of the various methods summarized above, as well as kits useful in performing such methods, and various other aspects of the invention, are described in more detail in the Detailed Description, Drawings, Examples, and Claims sections of this patent disclosure—each of which sections are intended to be read in conjunction with one another and in the context of the whole of this patent disclosure. Furthermore, one of skill in the art will recognize that the various embodiments of the present invention described throughout this patent disclosure can be combined in various ways, and that such combinations are within the scope of the present invention.

### BRIEF DESCRIPTION OF THE DRAWINGS

FIG. 1. Identification of germline mutations in ERCC3 in a family with multiple breast cancer cases. Sequencing was performed on 3 individuals affected with breast cancer, confirming identification of ERCC3 R109X in all 3 affected siblings. Pathology reports for individuals 11-3 and 11-5 showed both with well-differentiated (low grade) invasive ductal carcinoma diagnosed at Stage IA. One of the tumors was ER+PR+Her2+(II-3) the other one was ER+PR-Her2- (II-5).

FIG. 2A-F: Functional Evaluation of the ERCC3 mutant via overexpression in an ERCC3 deficient cell line. Transcript (FIG. 2A) and protein (FIG. 2B) levels in XPCS2BA parental cell line and cell lines stably overexpressing a lentiviral construct containing wild-type ERCC3 (WT) or the R109X mutant (R109X) cDNA. Expression from the mutant cDNA produces a truncated protein fragment of about 12 kDa. FIG. 2C-D—show relative cell viability of XPCS2BA and ERCC3 wild-type and mutant overexpressing cell lines at 72 hours following treatment with increasing doses of IlludinS (FIG. 2C) or UVC (FIG. 2D). FIG. 2E—shows the results of host cell reactivation assays showing reduced DNA repair ability of the mutant as opposed to wild-type ERCC3 overexpressing cell lines. Data represents the mean of three experiments with error bars representing the SD (FIG. 2C) or SEM (FIG. 2D, E). FIG. 2F—Phosphorylation of H2AX and Chk1 in response to UVC-induced DNA damage. XPCS2BA, WT or R109X cell lines were harvested at different time points following exposure to 20 J/m2 UVC and activation of H2AX and Chk1 was assessed by western blotting.

FIG. 3A-E: Modeling of ERCC3 R109X by CRISPR/Cas9 in a mammary epithelial cell line and functional analysis. ERCC3 transcript (FIG. 3A) and protein (FIG. 3B) levels in HMLE and CRISPR/Cas9 edited cells lines modeling the heterozygous R109X mutation. FIG. 3C—Relative cell viability of HMLE control and CRISPR edited cell lines at 72 hours following treatment with increasing doses of IlludinS. Data represents the mean of three experiments with error bars representing the SEM. FIG. 3D—Relative cell viability of HMLE control and combined CRISPR edited cell lines (with and without re-expression of the wild-type ERCC3) following treatment with IlludinS. FIG. 3E—Quantification of phosphorylated H2AX by flow cytometry in HMLE control and CRISPR edited cell lines harvested at different time points after DNA damage.

FIG. 4 Modeling ERCC3 R109X via CRISPR/Cas9 genome editing. Sanger sequencing performed on genomic DNA extracted from CRISPR/Cas9 edited HMLE cell lines harboring frameshift or point mutation at the ERCC3 R109 locus. The star indicates the nucleotide position c.325C within the chromatogram. The arrow indicates the position at which the CRIPSR/Cas9 mediated change occurs. The portion of sequence shown for P106fs (TTGCAGAGCCNATGNGCCNAN) is SEQ ID NO. 14. The portion of sequence shown for V107fs (AGAGCCAGTGAGCCAACCAAG) is SEQ ID NO. 15. The portion of sequence shown for T111fs (TGCCGACCAANCCNNGGNNNN) is SEQ ID NO. 16. The portion of sequence shown for R109x (GCCAGTGTGCNGACCAACCCA) is SEQ ID NO. 17.

FIG. 5 Haplotype analyses from the TAGC heterozygote carriers. Graphs of haplotype length are provided for R109X carriers (left hand graph—shown as “rs34295337”) and non-carriers (right-hand graph). Carriers of the R109X mutation showed a mean haplotype length of 3.4 cM which was significantly longer than non-carriers (p<10-14), suggesting a founder mutation.

FIG. 6A-B ERCC3 mutations present in the cBioPortal (MSKCC) datasets. (FIG. 6A) Lollipop plot indicating positions of mutations within the ERCC3 protein. Circles indicate mutations. The majority of mutations were missense mutations. Truncating mutations are shown by an asterisk (*) and different mutation types (not missense, not truncating) are shown with a hash (#). The arrow marks the R109X mutation. (FIG. 6B) Mutation frequencies of ERCC3 across different cancer types. Filter was applied to display only cancer types that show 1% or higher mutation frequency. The two bar graph portions that denote deletions are marked with an asterisk (*). The remaining bar graph portions represent other mutations.

FIG. 7 ERCC3 transcript levels in patient derived whole blood. Real time quantitative PCR of ERCC3 transcript (spanning exons 9 & 10). The relative transcript levels are reduced in the mutation carrier derived cDNA relative to a non-carrier sibling.

FIG. 8. Human ERCC3 amino acid sequence (accession number NP_000113.1). SEQ ID NO. 11. The arginine (R) at amino acid residue number 109 is shown in bold underlined text.

FIG. 9. Human ERCC3 nucleotide sequence (accession number NM_000122.1). SEQ ID NO. 12. Nucleotide positions 96 to 2444 comprise the ERCC3 open reading frame. The start (ATG) and stop (TGA) codons of the ERCC3 open reading frame are shown in bold underlined text. The cytosine (C) at nucleotide number 325 of the open-reading frame (position 420 of NM_000122.1) is also shown in bold underlined text.

FIG. 10. Human ERCC3 open-reading frame nucleotide sequence. SEQ ID NO. 13. This open reading frame sequence corresponds to nucleotides 96 to 2444 of SEQ ID NO. 12 (i.e. NM_000122.1). The cytosine (C) at nucleotide number 325 of the open-reading frame is shown in bold underlined text.

FIG. 11A-B. FIG. 11A—Results of experiments assessing the sensitivity of ERCC3 WT (HMLE+/+) and ERCC3 R109X heterozygous mutant (HMLE+/R109X) HMLE cell lines to Irofulven. The graph shows relative cell viability (plotted on the y axis) at each of the indicated Irofulven doses (plotted on the x axis). Data points represent the mean of three experiments with error bars representing the standard error of the mean (SEM). FIG. 11B—Results of experiments similar to those shown in FIG. 11A but performed using HMLE cell lines that also stably express h-RAS V-12—in addition to having the specified ERCC3 phenotype (i.e. either ERCC3 WT (HMLE+/+) or ERCC3 R109X heterozygous mutant (HMLE+/R109X). The graph shows relative cell viability (plotted on the y axis) at each of the indicated Irofulven doses (plotted on the x axis). Data points represent the mean of three experiments with error bars representing the SEM.

FIG. 12A-B. Results of experiments assessing the sensitivity of ERCC3 WT and ERCC3 R109X heterozygous mutant tumors to Irofulven in a mouse xenograft model. In all cases the tumors were hRAS-V12 positive. The graphs show tumor volume on the y axis plotted against days after treatment initiation on the x axis. FIG. 12A shows the data for the ERCC3 WT tumors, while FIG. 12B shows the data for the ERCC3 R109X mutant tumors.

FIG. 13. Real time PCR data using RNA extracted from cell either cell lines prior to xenograft injections (“cell line”), tumors grown from these cell lines in flanks of athymic nude mice (“vehicle”), or tumors grown from these cell lines in flanks of athymic nude mice under treatment with Irofulven (“Irofulevn”) Data was generated from two independent samples each with 3 technical replicates per experiment. Relative transcript level is plotted on the y axis. The 6 bars shown on the graph are—from left to right—WT cell line (black), WT vehicle (light gray), WT Irofulven (dark gray), R109X cell line (black), R109X vehicle (light gray), and R109X Irofulven (dark gray).

### DETAILED DESCRIPTION

While some of the main embodiments of the present invention are described in the above Summary of the Invention and in the Examples and Claims sections of this patent application, this Detailed Description section provides certain additional description relating to the compositions and methods of the present invention, and is intended to be read in conjunction with all other sections of the present patent application.

### Definitions and Abbreviations

As used herein the term “R109X mutation” refers to either: or (a) a mutation in the ERCC3 protein (i.e. the product of the ERCC3 gene) wherein the arginine (R) amino acid residue at position 109 has been replaced with a translation termination signal/stop codon (*)—such that the protein is truncated (i.e. a p.R109* protein mutation), or (b) a mutation in the ERCC3 gene wherein the cytosine (C) nucleotide at position 325 of the ERCC3 cDNA has been replaced with a thymine (T) (i.e. a c.325C>T DNA substitution) resulting in the creation of a stop codon. It will be clear from the context in which the term is used whether the gene, or the protein, or both, is/are referred to.

The above references to specific numbered amino acid residues (i.e. amino acid residue number 109 of the ERCC3 protein) and specific numbered nucleotide positions (i.e. nucleotide position 325 of the ERCC3 cDNA), are based on the human ERCC3s amino acid and nucleotide sequences SEQ ID NO. 11 (Genbank accession number NP_000113.1) and SEQ ID NO. 13, respectively. (SEQ ID NO. 13 is the human ERCC3 open-reading frame nucleotide sequence, and corresponds to nucleotides 96 to 2444 of SEQ ID NO. 12 (SEQ ID NO. 12 is Genbank accession number NM_000122.1)). However, it should be noted, and one of skill in the art will appreciate, that different sequences may have different numbering systems, for example, if they are man-made or naturally occurring variants of SEQ ID NO. 11 or 13, or if they are ERCC3 sequences from different species, or if a sequence is a genomic DNA sequence as opposed to a cDNA sequence, etc. By way of example, a human ERCC3 variant sequence could have amino acid residues added or removed as compared to SEQ ID NO: 11. As such, it is to be understood that, even though specific amino acid residues and/or nucleotide positions are referred to herein by their number, the description is not limited to only amino acids and nucleotides located at precisely that numbered position when counting from the beginning of a given amino acid sequence or a given nucleotide sequence, but, rather, that the “corresponding” amino acid residues or nucleotide positions in any and all ERCC3 sequences are intended—even if that residue is not at the same precise numbered position, for example if an ERCC3 sequence is shorter or longer than SEQ ID NO. 11 or 13, or has insertions or deletions as compared to SEQ ID NO. 11 or 13. One of skill in the art can readily determine what is the “corresponding” amino acid position or “corresponding” nucleotide position” to any of the specific numbered positions recited herein, for example by aligning a given ERCC3 amino acid sequence to SEQ ID NO. 11, or by aligning a given ERCC3 nucleotide sequence to SEQ ID NO. 13.

As used herein, the terms “about” and “approximately,” when used in relation to numerical values, mean within + or −10% of the stated value.

As used herein the abbreviation “WT” means wild type. Unless stated otherwise, and/or unless some other meaning is clear from the context in which the term is used, the term “WT” refers to sequences, cells, tumors, or subjects and the like that are wild type at ERCC3 amino acid position 109—i.e. that have, or encode, an arginine (R) at amino acid residue 109 of ERCC3—as opposed to having a R109X mutation.

Other abbreviations and terms are defined elsewhere in this patent specification, or else are used in accordance with their usual meaning in the art.

Active Agents

The methods and compositions provided by present invention involve various different active agents, including, but not limited to, DNA alkylating agents, such as Illudins.

As used herein the term “Illudin” is intended to refer to molecules in the Illudin class—including naturally occurring Illudin molecules and made-made analogues and derivatives thereof. The Illudins are a family of sesquiterpenes with antitumor and antibiotic properties produced by some mushrooms. In some embodiments the Illudin molecule used in accordance with the present invention is selected from the group consisting of: Illudin A, Illudin B, Illudin M, Illudin S, 6-Deoxyilludin M, dehydroilludin M, dihydroilludin M, 6-Deoxyilludin S, dehydroilludin S, dihydroilludin S and Irofulven. Irofulven (also known as 6-hydroxymethylacylfulvene, HMAF, and MGI-114) is a man-made analogue of Illudin S. The chemical structures of, and methods for the isolation and/or synthesis of, such Illudin molecules are known in the art. In addition, many of such molecules are commercially available. One of ordinary skill in the art will appreciate that, in addition to the various specified active agents referred to above or elsewhere herein, the compositions and methods of the present invention can also be carried out using analogues or derivatives of such specified active agents if, and provided that, such analogues and derivatives retain the key functional properties of the specified active agents. For example, one of ordinary skill in the art will appreciate that an analogue or derivative of a specified Illudin can be used provided that it retains DNA alkylating and/or antitumor activity, which can determined using methods known in the art and/or using one of the assays or methods described in the Examples section of this patent application.

Compositions

In certain embodiments, the present invention provides compositions, such as pharmaceutical compositions. The term “pharmaceutical composition,” as used herein, refers to a composition comprising at least one active agent as described herein, and one or more other components useful in formulating a composition for delivery to a subject, such as diluents, buffers, carriers, stabilizers, dispersing agents, suspending agents, thickening agents, excipients, preservatives, and the like.

For each of the embodiments described herein that involve use of or administration of an Illudin, use or administration of a pharmaceutical composition comprising an Illudin is also contemplated.

Subjects

As used herein the term “subject” encompasses all mammalian species, including, but not limited to, humans, non-human primates, dogs, cats, rodents (such as rats, mice and guinea pigs), cows, pigs, sheep, goats, horses, and the like—including all mammalian animal species used in animal husbandry, as well as animals kept as pets and in zoos, etc. However, in preferred embodiments the subjects are human. Breast cancer can occur in males and females, and the mutations that are described herein have been identified in both males and females. As such, in those embodiments involving breast cancer, as well as in all other treatment embodiments, the subject can be either a male or a female.

In some embodiments the subject has cancer, or is suspected of breast cancer. In some embodiments the subject has a tumor that has recurred following a prior treatment with other compositions or methods, including, but not limited to, chemotherapy, radiation therapy, or surgical resection, or any combination thereof. In some embodiments the subject has a tumor that has not previously been treated.

In some embodiments the subject may not have cancer but may be evaluated using one of the diagnostic methods described herein to determine if that subject is at risk of developing cancer, or has an increased risk of developing cancer.

In some embodiments the subject is of Ashkenazi Jewish ancestry.

In some embodiments the cancer is (or the subject has, or is suspected of having) breast cancer. In some embodiments the cancer is (or the subject has, or is suspected of having) colorectal cancer. In some embodiments the cancer is (or the subject has, or is suspected of having) NSCLC. In some embodiments the cancer is (or the subject has, or is suspected of having) bladder cancer. In some embodiments the cancer is (or the subject has, or is suspected of having) a glioma. In some embodiments the cancer is (or the subject has, or is suspected of having, a cancer that is) selected from the group consisting of breast cancer, colorectal cancer, NSCLC, bladder cancer, and glioma. In some embodiments the cancer is (or the subject has, or is suspected of having, a cancer that is) selected from the group consisting of colorectal cancer, NSCLC, bladder cancer, and glioma. In some embodiments the cancer is (or the subject has, or is suspected of having, a cancer that is) not breast cancer.

In some embodiments the subject has a mutation in the ERCC3 gene. In some embodiments the subject has a truncating and/or hypomorphic mutation in the ERCC3 gene. In some embodiments the subject has a truncating mutation in the region encoding the first putative helicase domain of the ERCC3 protein. In some embodiments the subject has a R109X mutation in the ERCC3 gene. In some embodiments the mutation in the ERCC3 gene is a germline mutation. In some embodiments the mutation in the ERCC3 gene is a de novo mutation. The mutation in the ERCC3 gene may be homozygous or heterozygous. Typically, the mutation is heterozygous.

In some embodiments the subject has an estrogen receptor-positive (ER+) breast cancer. In some embodiments the subject has an estrogen receptor-negative (ER+) breast cancer. In some embodiments the subject has a BRCA-negative breast cancer. In some embodiments the subject is of Ashkenazi Jewish ancestry and has an estrogen receptor-positive (ER+) breast cancer. In some embodiments the subject is of Ashkenazi Jewish ancestry and has a BRCA-negative breast cancer. In some embodiments the subject is of Ashkenazi Jewish ancestry, and has an estrogen receptor-positive (ER+) breast cancer, and has a BRCA-negative breast cancer.

Methods of Inhibiting Cancer Cell Proliferation and Methods of Treatment

In some embodiments the present invention provides methods for inhibiting the proliferation of cancer cells. Typically, such methods comprise contacting the cancer cells with an effective amount of an Illudin.

In some embodiments the present invention provides methods for treating cancer in a subject. Typically, such methods comrpise administering an effective amount of an Illudin to a subject.

As used herein, the terms “treat,” “treating,” and “treatment” encompass achieving a detectable improvement in one or more indicators of, or symptoms associated with, a cancer—such as one of the specific cancers and groups of cancers referred to herein. For example, such terms include, but are not limited to, inhibiting the proliferation of tumor cells, killing tumor cells, reducing the rate of growth of a tumor (or of tumor cells), halting the growth of a tumor (or of tumor cells), causing regression of a tumor (or of tumor cells), reducing the size of a tumor (for example as measured in terms of tumor volume or tumor mass), reducing the grade of a tumor, eliminating a tumor (or tumor cells), preventing, delaying, or slowing recurrence (rebound) of a tumor, improving symptoms associated with a tumor, improving survival from a tumor, inhibiting or reducing spreading of a tumor (e.g. metastases), and the like.

For each of the embodiments described herein as methods of treatment, it should be noted that corresponding/analogous methods of inhibiting the proliferation of cancer cells are also contemplated. Such methods of inhibiting the proliferation of cancer cells may involve inhibiting the proliferation of cancer cells in vivo and/or in vitro. For example, everywhere that a “method of treatment” is described herein, it is to be understood that the same (or analogous) method steps maybe employed in a method of inhibiting the proliferation of cancer cells. For example, where a method of treatment of a cancer is described that comprises administering an effective amount of an active agent to a subject, an analogous method of inhibiting the proliferation of such cancer cells comprising contacting the cancer cells with an effective amount of the active agent is also contemplated.

The methods of treatment provided herein typically comprise administering an effective amount of one of the active agents described herein (e.g. an Illudin, such as Irofulven) to a subject in need thereof (e.g. a subject having cancer, and typically a subject having an ERCC3 mutation, such as an R109X mutation).

In those methods that are directed to inhibiting the proliferation of cancer cells, such methods typically comprise contacting the tumor cells with an effective amount of one of the active agents described herein (e.g. an Illudin, such as Irofulven). Typically, the tumor cells have an ERCC3 mutation, such as an R109X mutation.

In some embodiments the present methods and compositions can be used to treat any cancer in a subject or to inhibit the proliferation of any cancer cell. In some embodiments the cancer is breast cancer. In some embodiments the cancer is colorectal cancer. In some embodiments the cancer is NSCLC. In some embodiments the cancer is bladder cancer. In some embodiments the cancer is a glioma. In some embodiments the cancer is selected from the group consisting of breast cancer, colorectal cancer, NSCLC, bladder cancer, and glioma.

In some embodiments the cancer is selected from the group consisting of colorectal cancer, NSCLC, bladder cancer, and glioma. In some embodiments the cancer not breast cancer.

In preferred embodiments the cancer or cancer cells has/have a mutation in the ERCC3 gene, such as, a truncating and/or hypomorphic mutation. In further preferred embodiments the the mutation is in the in the first putative helicase domain of the ERCC3 gene product. In further preferred embodiments the mutation is a R109X mutation.

As used herein the term “effective amount” refers to an amount of an active agent as described herein that is sufficient to achieve, or contribute towards achieving, one or more desirable outcomes, such as those described in the “treatment” description above. An appropriate “effective” amount in any individual case may be determined using standard techniques known in the art, such as in vitro or in vivo dose-response studies or dose escalation studies, and may be determined taking into account such factors as the desired use, desired route of administration (e.g. systemic vs. intratumoral), desired frequency of dosing, etc. Furthermore, an “effective amount” may be determined in the context of any co-administration method to be used. One of skill in the art can readily perform such dosing studies (whether using single agents or combinations of agents) to determine appropriate doses to use, for example using assays such as those described in the Examples section of this patent application—which involve administration of the agents described herein to cells or subjects (such as animal subjects routinely used in the pharmaceutical sciences for performing dosing studies).

For example, in some embodiments an effective amount of an active agent for use in the methods of the present invention may be calculated based on studies in humans or other mammals carried out to determine efficacy and/or effective amounts of the active agent. The effective amount may be determined by methods known in the art and may depend on factors such as pharmaceutical form of the active agent, route of administration, whether only one active agent is used or multiple active agents (for example, the dosage of a first active agent required may be lower when such agent is used in combination with a second active agent), and patient characteristics including age, body weight or the presence of any medical conditions affecting drug metabolism.

In some embodiments, one or more of the active agents is used at approximately its maximum tolerated dose, for example as determined in phase I clinical trials and/or in dose escalation studies. In some embodiments one or more of the active agents is used at about 90% of its maximum tolerated dose. In some embodiments one or more of the active agents is used at about 80% of its maximum tolerated dose. In some embodiments one or more of the active agents is used at about 70% of its maximum tolerated dose. In some embodiments one or more of the active agents is used at about 60% of its maximum tolerated dose. In some embodiments one or more of the active agents is used at about 50% of its maximum tolerated dose. In some embodiments one or more of the active agents is used at about 50% of its maximum tolerated dose. In some embodiments one or more of the active agents is used at about 40% of its maximum tolerated dose. In some embodiments one or more of the active agents is used at about 30% of its maximum tolerated dose. In some embodiments one or more of the active agents is used at about 20% of its maximum tolerated dose. In some embodiments one or more of the active agents is used at about 10% of its maximum tolerated dose.

One of the important features of the present invention, is that it has been found that cancer cells and cancers comprising the truncating hypomorphic R109X mutation in the ERCC3 gene are more sensitive to Illudin class molecules (and UV irradiation) than tumor cells lacking such a mutation. As such, lower doses of these agents can be used to inhibit the proliferation of cancer cells and/or to treat cancers in cells or subjects that have such mutations than would be required for cells or subjects not having such mutations. Given the high toxicity of some Illudins (39, 40, 41, 42), this is particularly advantageous, potentially reducing the occurrence and/or severity of unwanted side effects. For example, in some embodiments an Illudin may be administered to a subject at about 100%, or about 90%, or about 80%, or about 70%, or about 60%, or about 50%, or about 40%, or about 30%, or about 20%, or about 10%, of the dose of that Illudin that is effective for and/or approved for and/or typically used for the treatment of subjects not having the ERCC3 mutation. The Illudin Irofulven has been tested in numerous human clinical trials (39, 40, 41, 42). In some embodiments Irofulven may be administered to a subject at about 100%, or about 90%, or about 80%, or about 70%, or about 60%, or about 50%, or about 40%, or about 30%, or about 20%, or about 10%, of the dose of Irofulven that is effective for and/or approved for and/or typically used for the treatment of subjects not having an ERCC3 mutation. For example, Irofulven has been found to have a maximum tolerated dose in human clinical trials of 18 mg/m²/infusion, or 0.55 mg/kg/infusion, or 50 mg total per infusion (39, 40, 41, 42). Accordingly, in some embodiments the effective amount of Irofulven used in the methods of the present invention may be about 100%, or about 90%, or about 80%, or about 70%, or about 60%, or about 50%, or about 40%, or about 30%, or about 20%, or about 10%, of such maximum tolerated doses (i.e. of 18 mg/m²/infusion, or 0.55 mg/kg/infusion, or 50 mg total per infusion).

In carrying out the treatment methods described herein, any suitable method or route of administration can be used to deliver the active agents or combinations thereof described herein. In some embodiments systemic administration may be employed, for example, oral or intravenous administration, or any other suitable method or route of systemic administration known in the art. In some embodiments intratumoral delivery may be employed. For example, the active agents described herein may be administered either systemically or locally by injection, by infusion through a catheter, using an implantable drug delivery device, or by any other means known in the art.

In carrying out the treatment methods described herein, any suitable dosing schedule can be used to deliver the active agents or combinations thereof described herein. The Illudin Irofulven has been tested in numerous human clinical trials and suitable dosing schedules have been determined. In some embodiments such a dosing schedule may be employed. (See references 39, 40, 41, and 42 for doses and dosing schedules that may be employed. The contents of each of these references is hereby incorporated by reference). In some embodiments an Illudin (such as Irofulven) may be administered to a subject daily. In some embodiments an Illudin (such as Irofulven) may be administered to a subject weekly. In some embodiments an Illudin (such as Irofulven) may be administered to a subject biweekly. In some embodiments an Illudin (such as Irofulven) may be administered to a subject daily for a certain number of days (treatment “on” time)—followed by a period of days in which the Illudin is not administered to the subject (treatment “off” time). In some embodiments the treatment “on” and “off” periods may be repeated for multiple treatment cycles. For example, in some embodiments the treatment “on” time may be 1 day, 2 days, 3 days, 4 days, 5 days, 6 days, 7 days, 8 days, 9 days, 10 days, 11 days, 12 days, 13 days, or 14 days, or more. Similarly, the treatment “off” time may be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, or 24 days, or 25 days, or 26 days, or 27 days, or 28 days, or more. Similarly, the total “cycle” time (i.e. the “on” plus “off” time) may be 2 days, 3 days, 4 days, 5 days, 6 days, 7 days, 8 days, 9 days, 10 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days, or more. For example, in one embodiment an Illudin (such as Irofulven) may be administered to a subject daily for 5 days (treatment “on” time)—followed by a period of 16 days in which the Illudin is not administered to the subject (treatment “off” time), and this 21 day cycle may be repeated as many times as desired, e.g. once, twice, three times, four times, five times, etc. In another example, an Illudin (such as Irofulven) may be administered to a subject on day 1, not administered on days 2-7, administered again on day 8, and then not administered on days 9-28, and this 28-day cycle may be repeated as many times as desired, e.g. once, twice, three times, four times, five times, etc. In yet another example, an Illudin (such as Irofulven) may be administered to a subject on day 1, not administered on days 2-14, administered again on day 15, and then not administered on days 16-28, and this 28-day cycle may be repeated as many times as desired, e.g. once, twice, three times, four times, five times, etc. Numerous variations on such cycling dosages regimen are also possible.

In certain embodiments the compositions and methods of treatment provided herein may be employed together with other compositions and treatment methods known to be useful for cancer therapy, including, but not limited to, surgical methods (e.g. for tumor resection), radiation therapy methods, treatment with chemotherapeutic agents, treatment with antiangiogenic agents, or treatment with tyrosine kinase inhibitors. Similarly, in certain embodiments the methods of treatment provided herein may be employed together with procedures used to monitor disease status/progression, such as biopsy methods and diagnostic methods (e.g. MRI methods or other imaging methods).

For example, in some embodiments the agents and compositions described herein may be administered to a subject prior to performing surgical resection of a tumor, for example to shrink a tumor prior to surgical resection. In other embodiments the agents and compositions described herein may be administered both before and after performing surgical resection of a tumor.

In some embodiments the treatment methods described herein may be employed in conjunction with performing a diagnostic test to determine if the subject has, and/or has a tumor that comprises, cancer cells having, an ERCC3 mutation, such as a truncating/hypomorphic mutation, for example an R109X mutation.

Diagnostic Methods & Diagnostic Reagents

Several embodiments of the present invention comprise performing diagnostic methods and/or using diagnostic reagents to determine if cells or a subject have/has an ERCC3 mutation, such as an R109X mutation. The invention also provides kits for use in such methods and/or containing such diagnostic reagents. Such methods, reagents, and kits are useful in determining whether a subject is at risk for developing cancer and also in determining whether a subject is a candidate for treatment with an Illudin, or with a reduced dose of an Illudin.

Some of the diagnostic methods, reagents, and kits of the invention include, or involve using, a primer or probe that binds to the ERCC3 gene. In some embodiments the primer or probe is useful in sequencing the ERCC3 gene or in amplifying the ERCC3 gene or a portion thereof by PCR—such that one can determine whether or not the ERCC3 mutation is present. Exemplary sequencing primers include, but are not limited to, those comprising SEQ ID NO. 1 or SEQ ID NO. 2. Exemplary PCR primers include, but are not limited to, those comprising SEQ ID NO. 3, SEQ ID NO. 4, SEQ ID NO. 7, SEQ ID NO. 8, SEQ ID NO. 9, or SEQ ID NO. 10. Also included are primers and probes that are variants of such exemplary sequences, for example variants having greater than 70%, 80%, or 90% sequence identity to such sequences. In some embodiments, the variant sequences vary in length from the exemplary sequences provided herein. For example, the variant sequences may vary in length by 10 nucleotides, 9 nucleotides, 8 nucleotides, 7 nucleotides, 6 nucleotides, 5 nucleotides, 4 nucleotides, 3 nucleotides, 2 nucleotides, or 1 nucleotide as compared to the exemplary sequences provided herein. Such variant sequences may be used if they retain the desired functional properties, such as amplification of a region of an ERCC3 gene that comprises the site of the mutation or suspected mutation, or sequencing of a region of an ERCC3 gene that comprises the site of the mutation or suspected mutation, or detection of the mutation or suspected mutation.

Some of such methods, reagents, and kits include, or involve using, a primer or probe that binds differentially to nucleic acid molecules that comprise an ERCC3 mutation (such as the R109X mutation) and nucleic acid molecules that do not comprise the mutation.

Some of such methods, reagents, and kits include, or involve using, a mixture of primers or probes including both: (i) a primer or probe that binds selectively to nucleic acid molecules that comprise an ERCC3 mutation (such as the R109X mutation), and (ii) a primer or probe that binds selectively to nucleic acid molecules that do not comprise the mutation. In some of such embodiments one or both of such primers or probes are labeled with a detectable marker. Where both are labeled, each may be labeled with a different detectable marker. Such methods, kits, and reagents may be useful both in detecting the presence or absence of the mutation and determining whether one or both alleles are affected (i.e. whether the mutation is heterozygous or homozygous).

Some of the diagnostic methods, reagents, and kits of the invention include, or involve using, an anti-ERCC3 antibody. In some such embodiments any ERCC3 antibody that is capable of binding to one of the truncated ERCC3 mutant proteins described herein can be used. For example, such an antibody can be used in those embodiments where the existence of the mutation is to be detected based on the molecular weight of the ERCC3 protein (e.g. using a Western blotting technique). In some embodiments the antibody is one that binds differentially to ERCC3 proteins that comprise the R109X mutation and those that do not comprise the mutation. In some embodiments a mixture of antibodies is used including both (i) an antibody that binds to proteins that comprise the R109X mutation, and (ii) an antibody that binds to proteins that do not comprise the R109X mutation. In each embodiment the antibodies may be labeled with a detectable marker. Where more than one antibody is used, each may be labeled with different detectable markers.

Some of the diagnostic methods, reagents, and kits of the invention include, or involve using, a positive-control tissue sample, cell sample, nucleic acid sample, or protein sample, comprising the ERCC3 mutation, or a negative-control tissue sample, cell sample, nucleic acid sample, or protein sample, not comprising the ERCC3 mutation, or both of such controls (i.e. both the positive and negative controls).

### EXAMPLES

The invention is further described in this non-limiting “Examples,” as well as the Figures referred to therein.

Numbers in parentheses herein refer to the numbered references provided in the “Reference List” section of this application.

### Example 1

Known gene mutations account for approximately 50% of the hereditary risk for breast cancer. Moderate and low penetrance variants, discovered by genomic approaches, account for an as yet unknown proportion of the remaining heritability. As described further in this Example, a truncating mutation c.325C>T:p.Arg109* (R109X) in the ATP-dependent helicase ERCC3 was observed recurrently among exomes sequenced in BRCA negative, breast cancer-affected individuals of Ashkenazi Jewish ancestry. Modeling of the mutation in ERCC3 deficient or CRISPR/Cas9 edited cell lines showed a consistent pattern of reduced expression of the protein and concomitant hypomorphic functionality when challenged with UVC exposure or treatment with the DNA alkylating agent IlludinS. Overexpressing the mutant protein in ERCC3-deficient cells only partially rescued their DNA repair-deficient phenotype. Comparison of the frequency of this recurrent mutation in over 6500 chromosomes of breast cancer cases and 6800 Ashkenazi controls showed a significant association with breast cancer risk (OR_{BC}=1.53, OR_{ER+}=1.73) particularly for the estrogen receptor positive (ER+) subset (P<0.007).

Genetic susceptibility to breast cancer has been shown to be strongly associated with rare coding gene mutations and common non-coding genomic variants (1). Loss of function mutations in well characterized genes, such as those in BRCA1/2 and other members of the homologous recombination pathway, are routinely used for clinical risk assessment in cancer, but account for a small fraction of excess familial risk (2). Common single nucleotide polymorphisms (SNPs) are yet to demonstrate clinical utility, but over 90 SNPs account for over 37% of familial relative risk (3). An as yet to be defined proportion of the remaining familial risk is accounted for by variants of “moderate” risk, including coding or non-coding variants in the DNA repair pathways such as nucleotide excision repair (NER), mismatch repair (MMR) and base excision repair (BER), which play an important role in checkpoint and genomic integrity maintenance (4, 5). In addition to serving as candidate cancer susceptibility genes, members of the NER pathway also play a critical role in DNA damage repair caused by chemotherapeutic agents and radiation exposure. ERCC3 is an ATP dependent DNA helicase that is part of the TFIIH transcription factor complex. In this report, we characterize the functions and demonstrate an association of a recurrent founder mutation in ERCC3 with breast cancer risk.

Results

Identification of a Recurrent Ercc3 R109x Mutation in Ashkenazi Probands with Brca1/2 Negative Breast Cancer

Exome sequencing was carried out on DNA extracted from peripheral blood/saliva of 46 early onset (<45 years) and 13 familial BRCA wild type breast cancer probands (33 with known ER positive status) of Ashkenazi Jewish ancestry. We filtered for rare protein truncating variants using public data sources such as ESP, 1000 genomes and ExAC without TCGA. We further filtered for recurrent mutations in the DNA repair genes with low background mutation load using the smallest RVIS percentage (6) amongst 70 genes, calculated based on the ExAC data. We observed three individuals in the same BRCA wild type kindred (FIG. 1) with the same protein truncating mutation, R109X in ERCC3 (HGMD: c.325C>T: p.R109*; chr2:128050332; rs34295337). Sanger sequencing was used to confirm the next generation sequencing findings. Two of the siblings were affected with estrogen receptor positive breast cancer and the third was a male with breast cancer. The same mutation was also observed in two other individuals at relatively young age (<40 years), from unrelated kindreds with multiple cases of breast cancer. Details of the families and cancer incidence is described in Table 2. The variant is almost absent in other world populations, while relatively rare in Caucasians in public data sources such as ExAC Consortium (Non-Finnish EUR MAF=0.0008, ESP-EUR MAF=0.0007). Analysis of the structure of the first 300 amino acids of ERCC3 showed that R109 is most likely part of a right handed alpha helix (a Ramachandran plot of the first 300 amino acids of ERCC3 showed that the Arg109 residue was within the left lower quadrant corresponding to the right handed alpha-helix as predicted by the ab-initio modeling server EVfold.)

Phenotype Rescue by Complementation Assay

To understand whether this variant has a deleterious effect on the gene function, we carried out a series of in vitro functional assays. Using a previously well characterized ERCC3 deficient cell line derived from an XP/CS patient (7), phenotype rescue after DNA damage was assessed by cell viability assay. The parental XPCS2BA cell line and derived lines stably overexpressing the ERCC3 R109* mutant (R109X), or the wild type (WT) (FIG. 2A & FIG. 2B) showed varying degrees of sensitivity to DNA damage inducing agents such as UVC and a fungal sesquiterpene IlludinS. We observed that cells overexpressing ERCC3 R109X showed significantly higher cell death compared to the ERCC3 WT, while the untransfected cells all died upon exposure to higher doses of both IlludinS and UVC (FIG. 2C and FIG. 2D). Exposure at 2 ng/ml of IlludinS (FIG. 2C), a fungal toxin from the Jack O′lantern mushroom (Omphalotus illudens) that creates DNA damage which blocks transcription with demonstrated antitumor activity (8), showed that 3%, 42% and 77% of the XPCS2BA, R109X and WT cells survived, respectively (P<0.0001). At a dose of 4 ng/ml, most of the WT cells survived, demonstrating that the mutant cells were transiently and partially able to withstand UV induced DNA damage, while failing to adequately repair DNA damage to ensure survival. Similarly, following UVC irradiation at a dose of 10 J/m², 72 hours post-exposure, 42% of the R109X cells survived compared to 63% of WT cells (P<0.0001). At a dose of 4 ng/ml, these numbers reduced more drastically for the mutant, showing the same trend as the IlludinS exposure experiment. (FIG. 2D).

Host Cell Reactivation Assay

Using an exogenous source of DNA, namely a luciferase reporter plasmid, we measured the ability of intact live cells to repair DNA damage. Through measurement of the reporter, the extent of reactivation of the damaged plasmid is quantitated and is a readout of DNA repair activity. Relative luminescence measured after co-transfection of the reporter plasmid (exposed to 600 J/m²) showed a 1.5 fold difference between mutant and WT overexpression cell lines, suggesting a markedly reduced reporter reactivation in the R109X (FIG. 2E) (P<0.0001). We observed almost no reactivation in the parental cell line at this dose.

Activation of the DNA Damage Response Pathway Following UVC Irradiation

DNA damage response is marked by activation of H2AX (γ-H2AX) and leads to the induction of cell cycle checkpoint initiation by activation of Chk1. To explore the ability of ERCC3 R109X to trigger the checkpoint cascade, in response to DNA damage, we examined γ-H2AX and phosphorylated Chk1 following UVC-mediated DNA damage induction in XPCS2BA cells. UV-induced phosphorylation of H2AX is reduced in cells deficient in the nucleotide excision repair pathway (9). In agreement with this, we find lower levels of activated H2AX in the XPCS2BA cell line compared to cells where WT ERCC3 expression has been restored. In the mutant, we observed lower γ-H2AX levels, almost as low as in the parental cell line. Activation of the checkpoint kinase Chk1 is also strongly reduced in the mutant as compared to the WT overexpressing cells, and similar to the untransfected XPCS2BA (FIG. 2F).

Functional Characterization of Genome Edited ERCC3 Mutants in Human Mammary Epithelial Cells

Overexpression of ERCC3 mutant constructs does recapitulate physiological levels of protein in a germline heterozygous mutation state within cells. Therefore, using CRISPR/Cas9, we engineered several heterozygous mutations in the mammary epithelial cell line HMLE that mimic the site of the originally discovered recurrent mutation in breast cancer individuals (Table 3, FIG. 4). Quantitative real time PCR of ERCC3 transcripts (Exons 1, 2 and Exons 9, 10) showed relative transcript levels reduced by half in these cells when compared to the parental HMLE cell line (FIG. 3A). Western blotting also showed the reduction in total ERCC3 protein levels (FIG. 3B) suggesting that in the CRISPR clones, ERCC3 is transcribed mainly from the remaining WT allele. Since we did not observe any homozygous mutations amongst the surviving CRISPR clones, we are unsure if a homozygous mutation is viable. The ERCC3 CRISPR clones generally showed substantial reduction in relative cell viability 72 hours after exposure to IlludinS (FIG. 3C). At 2 ng/ml IlludinS, all the parental HMLE cells survived, while the CRISPR edited cell lines showed significantly reduced survival (P<0.0001). These data suggest that the mutants function at a much lower efficiency compared to WT and hence may be classified as hypomorphs. To demonstrate that the observed effect of IlludinS on the viability of the CRISPR edited HMLE is specific to the resulting ERCC3 deficit, we performed rescue experiments by overexpressing WT ERCC3 in these cells. We observed that the ERCC3 CRISPR cell lines, after stable overexpression of WT ERCC3, showed the same response to IlludinS as the WT HMLE cell line (FIG. 3D), thereby showing complete rescue of the phenotype confirming that this phenotype was indeed a result of the engineered ERCC3 deficiency.

To assess the induction and removal of phosphorylated H2AX in response to DNA damage we performed immunostaining and flow cytometric analysis of γ-H2AX positive cells following treatment with IlludinS. While this led to a similar fold induction of γ-H2AX positive cells in the wild type and CRISPR clones (not shown), the reduction in γ-H2AX was significantly lower in the ERCC3 CRISPR mutants, indicating a less efficient DNA damage repair in these cells (P<0.05) (FIG. 3E).

ERCC3 R109X as a Risk Allele for Breast Cancer in Ashkenazim

Using breast cancer affected individuals and controls from the Memorial Sloan Kettering Cancer Center, the University of Pennsylvania and the Clalit National Israeli Cancer Control Center, we performed a matched case control association of the ERCC3 variant using Taqman genotyping and sequencing across 3286 cases and 2716 controls. A third group of 705 controls were sourced from The Ashkenazi Genome Consortium (TAGC) project [(10) and in preparation] control resource (Table 1A). All control groups showed similar allele frequencies. 101 heterozygote carriers were present in the combined data. A 1.53-fold increased risk was observed for breast cancer (OR=1.53, lower CI=1.07; P=0.023) after examining over 6500 chromosomes in cases and 6800 chromosomes in controls (Table 1A). A stronger association was observed with the ER positive subtype (OR=1.73, lower CI=1.19, P=0.007) (Table 1B). The majority of the tumors in the carriers had an ER positive status. These data show that the ERCC3 c.325 T-allele is a moderate risk factor for breast cancer in individuals of Ashkenazi ancestry. “Unphased haplotype analyses from the TAGC heterozygote carriers suggested a founder mutation (FIG. 5). In the chromosomal region 2q14.3, a 3.4 cM long haplotype was observed in the carriers which was significantly longer than in non-carriers (P<10⁻¹⁴).

TABLE 1A

| Center | Cases | Controls | Ca-Alt | Ca-Ref | Ctrl-Alt | Ctrl-Ref | P | OR | Lower-CI |
|---|---|---|---|---|---|---|---|---|---|
| MSKCC/ | 1968 | 1129 | 31 | 3905 | 10 | 2248 | | | |
| PENN | | | | | | | | | |
| CLALIT | 1318 | 1587 | 29 | 2607 | 22 | 3152 | | | |
| TAGC | 0 | 705 | 0 | 0 | 9 | 1401 | | | |
| Total | 3286 | 3421 | 60 | 6512 | 41 | 6801 | 0.023 | 1.53 | 1.07 |

TABLE 1B

| Center | Cases | Controls | Ca-Alt | Ca-Ref | Ctrl-Alt | Ctrl-Ref | P | OR | Lower-CI |
|---|---|---|---|---|---|---|---|---|---|
| MSKCC/ | 1288 | 1129 | 23 | 2553 | 10 | 2248 | | | |
| PENN | | | | | | | | | |
| CLALIT | 992 | 1587 | 24 | 1960 | 22 | 3152 | | | |
| TAGC | 0 | 705 | 0 | 0 | 9 | 1401 | | | |
| Total | 2280 | 3421 | 47 | 4513 | 41 | 6801 | 0.007 | 1.73 | 1.19 |

TABLE 2

| Family | Cancer | | Age | Ancestry/ | | ER | PR | HER-2 | |
|---|---|---|---|---|---|---|---|---|---|
| ID | Diagnosis | Histology | of Onset | Religion | Family History | Status | Status | Status | Gender |
| 48732 | Breast | Lobular | 52 | Ashkenazi | Mother: Colorectal at 61; | Positive | Negative | Negative | Unknown |
| | | and | | Jewish | Matemal Aunt: Breast at 74 | | | | |
| | | Ductal | | | | | | | |
| 48346 | Breast | Lobular | 64 | Ashkenazi | Mother Melanoma at 53; | Positive | Negative | Negative | Unknown |
| | | | | Jewish | Matemal Aunt: Colorectal | | | | |
| | | | | | at 70; Paternal Mother: | | | | |
| | | | | | Early-onset Breast | | | | |
| 47704 | Breast | Ductal | 54 | Ashkenazi | Mother: Breast at 82; | Positive | Positive | Negative | Unknown |
| | | | | Jewish | Matemal cousin: Breast at | | | | |
| | | | | | 52; Father: Colorectal at 80; | | | | |
| | | | | | Paternal Uncle: Lung at 50; | | | | |
| | | | | | Paternal Aunt: “Blood | | | | |
| | | | | | Cancer” at 71 | | | | |
| 47473 | Breast | Ductal | 52 | Ashkenazi | Mother: Breast at 41, | Positive | Negative | Positive | Unknown |
| | | | | Jewish | Colorectal and Melanoma | | | | |
| | | | | | age UNK; Maternal cousin: | | | | |
| | | | | | Breast at 30 | | | | |
| 42836 | Breast | Lobular | 52 | Ashkenazi | Mother: Breast at 68; | Unknown | Unknown | Unknown | Unknown |
| | | and | | Jewish | Matemal Uncle: Prostate at | | | | |
| | | Ductal | | | 65 | | | | |
| 44371 | Breast | Ductal | 57 | Ashkenazi | Mother: Breast at 45; | Positive | Positive | Negative | Unknown |
| | | | | Jewish | Father: Pancreas at 85 | | | | |
| 44965 | Breast | Ductal | 72 | Ashkenazi | Mother: Breast at 78; Sister: | Unknown | Unknown | Unknown | Unknown |
| | | | | Jewish | Stomach at 69; Maternal | | | | |
| | | | | | Aunt: Breast at 60 | | | | |
| 44997 | Breast | Ductal | 70 | Ashkenazi | Brother: Prostate at 70; | Positive | Positive | Positive | Unknown |
| | | | | Jewish | Paternal Aunt: *CSU; | | | | |
| | | | | | Paternal Aunt: *CSU; | | | | |
| | | | | | Niece: Breast at 37; Niece: | | | | |
| | | | | | Lymphoma age UNK; | | | | |
| | | | | | Niece: *CSU | | | | |
| 44618 | Breast | Ductal | >80 | Ashkenazi | Mother: Breast at 75, | Positive | Positive | Positive | Unknown |
| | | (Bilateral) | | Jewish | Bladder at 85; Brother: | | | | |
| | | | | | Lung at 83; Maternal | | | | |
| | | | | | Grandmother: *CSU at 76 | | | | |
| 45678 | Breast, | Lobular | 60 | Ashkenazi | Maternal Aunt: Colorectal; | Unknown | Unknown | Unknown | Unknown |
| | Colorectal | | | Jewish | Niece: Breast <60 | | | | |
| 45759 | Breast | DCIS | 59 | Ashkenazi | Sister Breast at 65; Sister: | Positive | Positive | Unknown | Unknown |
| | | | | Jewish | Lymphoma age UNK; | | | | |
| | | | | | Brother: Liver at 55 | | | | |
| 57546 | Control | N/A | N/A | Unknown | Matemal Sister: Esophagus | N/A | N/A | N/A | Unknown |
| | | | | | at 78 | | | | |
| 53103 | Control | N/A | N/A | Muslim | No significant family | N/A | N/A | N/A | Unknown |
| | | | | | history | | | | |
| 53158 | Control | N/A | N/A | Unknown | Mother: Breast at 52, Eye at | N/A | N/A | N/A | Unknown |
| | | | | | 31; Patemal Grandmother: | | | | |
| | | | | | Breast at 70; Paternal Aunt: | | | | |
| | | | | | Bone at 51 | | | | |

TABLE 3

| | | Mutation | AA | Stop |
|---|---|---|---|---|
| Clone | Mutation type | position | change | codon AA |
| P106fs | delTGCCG | c.318 | P106fs | 112 |
| V107fs | delAGTGTGCC | c.321 | V107fs | 113 |
| T111fs | insA | c.331 | T111fs | 115 |
| R109X | C > T | c.325 | R109X | 109 |

### DISCUSSION

Here, we identified a truncating germline mutation in the first putative helicase domain of the DNA repair gene ERCC3 and report a strong genetic association with risk of breast cancer in individuals of Ashkenazi Jewish (AJ) ancestry. The R109X variant, seen in 1.83% of AJ breast cancer individuals and 2.06% in the ER+ subset, confers moderate risk (OR_{BC}=1.53; OR_{ER+}=1.73). The same mutation was also observed twice as a somatic event in a breast carcinoma and a soft tissue sarcoma (FIG. 6A). Mutations within the ERCC3 helicase domain are also often seen in tumors such as melanoma, in addition to non-small cell lung cancer, colorectal, esophagogastric and bladder cancers. In general, ERCC3 is seldom disrupted in somatic tissue by genomic integrity loss such as amplification or deletions (FIG. 6B). In the LOVD database, the variant was seen in 6/8600 individuals of European ancestry, a similar frequency to public databases. As a core component of the TFIIH basal transcription factor, ERCC3 ATP-dependent DNA helicase has key functions in both RNA transcription by RNA polymerase II and in nucleotide excision repair (NER) following DNA damage (11). It has previously been associated with Mendelian DNA repair disorders such as xeroderma pigmentosum (XP), combined xeroderma pigmentosum/cockayne syndrome (XP/CS) and trichothiodystrophy (TTD) with hallmarks of increased photosensitivity, cancer predisposition and impaired development (12, 13). Hypomorphic mutations in the DNA damage repair gene NBN cause the autosomal recessive chromosomal instability disorder Nijmegen breakage syndrome (NBS) in homozygous individuals, but also have been shown to lead to increased cancer incidence in heterozygous relatives of NB S patients thus providing a plausible example of a hypomorphic mutation that is also a susceptibility gene (14, 15). Similarly, ERCC3 R109X behaves as a hypomorph in our functional assays. In experiments using an ERCC3 deficient cell line, R109X was partially successful at phenotype rescue, with the cells exhibiting intermediate repair capability, suggesting that they are more likely to harbor a second hit following genotoxic events. Importantly, ERCC3 transcript expression was shown to be lower in the proband's cells compared to a non-mutation carrier (FIG. 7) and electropherograms of genomic DNA and cDNA showed reduced peaks of the mutant allele.

Dosage-dependency of key molecular components of the DNA damage repair pathway, such as haploinsufficiency, has been previously demonstrated (16-20), leading to tumorigenesis in vitro and in vivo models of H2AX, BLM and CHEK1.

ERCC3 homozygous knockout mice have been shown to be embryonic lethal, indicating that the gene is necessary for development (21). Additionally, very few ERCC3 mutations have been reported, even amongst XP patients, suggesting the gene is intolerant to common mutational mechanisms. Analysis of gene based mutability from the ESP and ExAC exome data have shown that quantitative metrics that predict gene-conservation and mutation tolerance rank ERCC3 as bearing low background mutational load (22). In light of the rarity of known mutations in the ERCC3, there have thus far been no studies elucidating cancer susceptibility in heterozygous carriers. The only mouse model that has been described modeled the XP/CS hereditary DNA repair deficiency syndromes (21). Heterozygous knockout animals of NER genes XPC and XPE/DDB2 showed increased cancer incidence after exposure to UV irradiation (23, 24), while heterozygous XPC knockout mice also showed elevated frequency of spontaneous mutations as a function of age (25). Also, a mouse model harboring a mutation in the other helicase of the TFIIH complex, 6°) D, shows a strongly increased cancer incidence in response to UV exposure (26).

This is the first study that shows a specific truncating mutation in ERCC3 conferring increased risk to breast cancer. However, polygenes in DNA repair and/or other pathways, acting epistatically with ERCC3, are likely contributors to and modifiers of the heritable risk of breast cancer, complicating attempts to demonstrate co-segregation of single variants in multiplex kindreds. We have previously demonstrated a modestly elevated breast cancer risk associated with specific mutations of CHEK2 and APC in the Ashkenazi Jewish genetic isolate (27, 28) and other recurring mutations of CHEK2, HOXB13, PALB2, and RAD51C have been variably associated with breast cancer risk in diverse populations (29-32). While more frequent in Ashkenazi Jews, we have observed ERCC3 R109X as well as other ERCC3 mutations in non-Ashkenazi individuals (data not shown). The enhanced susceptibility of ERCC3 mutant cells to reagents of the fungal sesquiterpene class, demonstrated here, suggests e that such agents may have increased therapeutic efficacy in an ERCC3 mutant genetic background.

Materials & Methods

Next Generation Sequencing of Breast Cancer Probands and Controls

One microgram of germline DNA extracted from peripheral blood was used for whole exome capture using the Agilent SureSelect 38 Mb paired-end sequencing with the Illumina HiSeq 2000. Sequence reads in the form of FASTQ files were aligned to the human decoy reference (GRCh37) to generate BAM files using BWA v0.7.12. Picard tools was used for quality metric calculation and marking duplicate reads. The Genome Analysis Tool Kit (GATK) version 3.3.0-g37228af was used for variant calling using the haplotype caller algorithm. Variant quality score recalibrated (VQSR) data was used for filtering variants. Variant level and interval level annotations were performed using SNPEff, ANNOVAR, CAVA programs. Downstream analysis consisted of filtering out low quality variant calls and those already reported as common in public databases.

The TAGC (The Ashkenazi Jewish Genome Project) has sequenced the whole genomes of 128 and 577 individuals of AJ ancestry using the Complete Genomics and Illumina X10 sequencing platform at the New York Genome Center, respectively. The paired end libraries were generated using TruSeq DNA Nano kit, sequenced to an average depth of 30 and reads were aligned using a standard pipeline involving BWA version 0.7.8 and GATK version 3.2.2. Extensive QC is performed on all WGS samples, including alignment rates (>97%), median and mean library insert size (>350 bp), percent duplication (typically <20%), mean genome coverage (>30×) and uniformity of coverage, TiTv and Het-Hom ratios. An automated concordance check is also performed against the SNP array genotyping data, to ensure against sample swap at any step during the sequencing process, and to further validate the quality of the SNV calls from the sequencing data.

All research participants provided written consent to an IRB-approved research protocol to allow for the collection and use of biospecimens.

Haplotype Analysis

Using only high quality bi-allelic SNPs, the haplotype lengths carrying the ERCC3 R109X mutation were calculated to either side of the mutation in the TAGC carriers as the length until an opposite homozygous genotype. This is an overestimate, due to the lack of accurate phasing information. The mean length was 3.4 cM, estimated by using the Hapmap recombination rates for the region. The mean lengths of haplotypes shared between noncarriers were also computed. The significance of the difference between the distributions of haplotype lengths at the carriers and at 100 random non-carriers was calculated using the Kolmogorov-Smirnov test.

Sanger Sequencing

Sanger Sequencing of the ERCC3 R109X variant was performed using the following primers.

ERCC3_F1:
(SEQ ID NO. 1)
CATGGAGCACCTATGCCTATT
ERCC3_R1:
(SEQ ID NO. 2)
CTGCAACTCATGTTTCCTTGTC

Taqman Genotyping

The allelic discrimination assay C_25963434_10 for genotyping was done using Taqman (Life Technologies, Carlsbad, Calif.). The assay was run on an ABI HT7900 machine and automatically clustered and manually reviewed. Confirmed heterozygotes were run as positive controls and duplicate concordances checked per plate.

Statistical Analyses

Allele counts were tabulated from the Taqman genotyping after QC. Statistical analysis was performed using R statistical package using the fisher.test module. Since, the truncating genetic variant was hypothesized and shown by functional assays to lead to increased risk associated with reduced DNA repair efficiency, we report one-sided Fisher's exact test results. Nevertheless, two sided tests for both breast cancer and ER status were also significant at 5% level (BC overall: P=0.036, OR=1.53, 95% CI=1.01-2.34; ER+: P=0.011, OR=1.73, 95% CI=1.11-2.70).

Plasmids

The pENTR221 plasmid containing human ERCC3 ORF was purchased from TransOMIC (Huntsville, USA). The ERCC3 ORF was cloned into the pLX302 lentiviral expression plasmid, a gift from David Root (Addgene #25896). The ERCC3 R109X mutant was generated from the WT ERCC3 plasmid using the QuickChange II XL Site-Directed Mutagenesis Kit (Agilent). pSpCas9(BB)-2A-GFP (PX458) was a gift from Feng Zhang (Addgene #48138). Guide RNAs were designed using the CRISPR Design Tool (37). The 24-mer oligonucleotides were synthesized (37) (Integrated DNA technologies, Coralville, USA), annealed and cloned into pX458.

Cell Culture and Transfections

The HMLE cell line was \ grown in mammary epithelial growth medium (MEGM) and supplements as recommended by Lonza. The XPCSBA-sv40 cell line was grown in RPMI 1640-HEPES medium (Invitrogen), supplemented with 10% FBS and 1% penicillin-streptomycin and Glutamate. Hek293T cells (ATCC, CRL-3216) were maintained in Dulbecco's Modified Eagle's Medium (DMEM) supplemented with 10% FBS and 1% penicillin-streptomycin and Glutamate. Cell cultures were maintained in a humidified incubator at 37° C. in 5% CO2 and tested for mycoplasma on a monthly basis.

Transfections were carried out with the Amaxa® Cell Line Nucleofector® Kit V (Lonza, Walkersville) and Nucleofector IIb device according to the manufacturer's instructions. Viral vectors, co-transfected with psPAX2 and pseudotyped with VSV-G were produced in Hek293T cells using Lipofectamin2000 transfection reagent. The virus supernatant was concentrated by centrifugation for 90 minutes at 20,000 RPM at 4° C. and pellets were dissolved in OptiMEM (Gibco). Transduction of cells with virus supernatant was carried out in the presence of 8 μg/ml Polybrene. Stably transfected cell lines were generated by selection with 0.5 μg/ml puromycin.

Cells co-transfected with pmaxGFP (Lonza) and the pX458-sgRNA plasmids, containing guide RNAs targeting the ERCC3 locus in close proximity to the ERCC3 mutation site (chr2: 128050332) with or without a repair template (ssODN) containing the specific mutation, were sorted as single cells into 96 well plates based on GFP fluorescence using a BD FACSAria™ cell sorter.

Screening of Crispr/Cas9 Edited Cell Lines

Single cell clones generated from the transfection mentioned above were expanded and genomic DNA extracted using the QuickExtract DNA Extraction Solution (Epicentre, Madison, Wis.). The genomic region surrounding the target site was amplified using the following primer sequences:

ERCC3_SURV F,
(SEQ ID NO. 3)
5′- TGTGGTGTTGGGCAGCTTAT-3′
ERCC3_SURV R,
(SEQ ID NO. 4)
5′- ACACTCACTTTGGGCTGCAT-3′

The purified PCR products were subjected to Sanger sequencing.

TABLE 4
Primer/Probe Sequences

| Name | Sequence (5′ to 3′) | SEQ ID NO. |
|---|---|---|
| ERCC3_F1 | CATGGAGCACCTATGCCTATT | SEQ ID NO. 1 |
| ERCC3_R1 | CTGCAACTCATGTTTCCTTGTC | SEQ ID NO. 2 |
| ERCC3_SURV F | TGTGGTGTTGGGCAGCTTAT | SEQ ID NO. 3 |
| ERCC3_SURV R | ACACTCACTTTGGGCTGCAT | SEQ ID NO. 4 |
| RPL32 F | CATCTCCTTCTCGGCATCA | SEQ ID NO. 5 |
| RPL32 R | AACCCTGTTGTCAATGCCTC | SEQ ID NO. 6 |
| ERCC3 F1 | GTCCGCGAAGATGACAAAATTG | SEQ ID NO. 7 |
| ERCC3 R1 | AATTCAGGAGACATAGGGCAC | SEQ ID NO. 8 |
| ERCC3 F3 | ATGGGCAAAAGAGACCGAG | SEQ ID NO. 9 |
| ERCC3 R3 | CTGACTCATCCACCTGCTTC | SEQ ID NO. 10 |

Real-Time PCR

RNA was extracted 24 hours after transduction using the RNeasy Mini Kit (Qiagen) and reverse transcribed with the ReadyScript cDNA Synthesis Mix (Sigma-Aldrich). Quantitative Real-time PCR analyses were performed on an ABI PRISM 7900HT Sequence Detection System using the Power SYBR® Green PCR Master Mix (Life Technologies) according to the manufacturer's instructions. Following initial incubation for 10 min at 95° C., amplification was performed for 40 cycles at 95° C. for 15 sec and 60° C. for 1 min. The RPL32 gene was used as the internal standard. Analysis was performed based on the comparative CT method. Values reported are mean of triplicate experiments. The following primer sequences were used: RPL32 F (SEQ ID NO. 5), RPL32 R (SEQ ID NO. 6), ERCC3 F1 (SEQ ID NO. 7), ERCC3 R1\ (SEQ ID NO. 8), ERCC3 F3 (SEQ ID NO. 9), and ERCC3 R3 (SEQ ID NO. 10)

Western Blotting

Protein lysates were prepared in RIPA buffer (Pierce). Samples were run on 4-12% gradient Bis-Tris SDS-PAGE gels (Invitrogen), transferred onto PVDF membranes (Bio-Rad) and probed with antibodies against ERCC3 (ARP37963_P050; 1:2,500; Aviva Systems Biology), phospho-Histone H2A.X (Ser139) (1:1000; Cell Signaling Technology), phospho-Chk1 (Ser345) (1:1000; Cell Signaling Technology) and GAPDH (V-18; 1:400; Santa Cruz Biotechnology). HRP-conjugated secondary antibodies were detected using ECL Prime Western Blotting Detection Reagent (GE Healthcare).

Cell Viability Assays

For viability assessment following treatment with IlludinS (Cayman Chemical), cells were seeded into 96 well plates 24 hours prior to treatment. For post-UV viability assays, cells were exposed to the appropriate doses of UV irradiation and subsequently seeded into 96 well plates. Cell viability was measured after 72 hours using the CellTiter-Glo® Luminescent Cell Viability Assay (Promega).

Host Cell Reactivation Assay

The pISO plasmid, derived from the pGL3-control vector containing the Firefly luciferase gene (Promega, Madison, Wis.) and the pIS2, a derivative from the pRL-SV40 vector (Promega) containing the Renilla luciferase gene (as an internal transfection control) were used for transfection of cells. The pISO vector was treated with increasing doses of UVC radiation. These vectors were co-transfected into the XPCS2BA and stable ERCC3 WT and R109X overexpressing cell lines using the Fugene 6 Transfection Reagent (Promega, Madison, Wis.). Forty-eight hours after DNA transfection, the luciferases' activity was measured with the Dual-Glo Luciferase Assay System (Promega) and a GloMAX Luminometer (Promega).

Flow Cytometry

For flow cytometric analysis, 24 h after seeding, cells were either left untreated or were treated with 8 ng/ml IlludinS for 1 h. Treated cells were subsequently washed with PBS and supplemented with drug-free medium. The cells were harvested at different time points after treatment and fixed with 4% PFA for 10 min at RT. Cells were quickly chilled on ice and ice-cold MeOH was added to a final concentration of 90%. The cells were incubated on ice for 30 min and stored at −20° C. For immunostaining the cells were washed twice with PBS/0.5% BSA and incubated with anti phospho-Histone H2A.X (Ser139) Antibody (1:250; Cell Signaling Technology) for 1 h at RT, washed again and incubated with Alexa-488 conjugated secondary antibody for 45 min at RT. For each condition 20.000 cells were recorded and data from two replicate experiments was analyzed using the FlowJo software (V10.1).

### Example 2

Unless otherwise stated, all materials and methods used in the experiments described in this Example were as for Example 1.

A “case-control” analysis of the ERCC3 R109X mutation was performed using 12-245 IMPACT germline sequencing data. The “cases” analyzed were a subset of individuals who self-identified as Ashkenazi Jews in a cohort set of 9000 individuals. The gNOMAD AJ cohort was used as the “controls” in this case-control analysis. The results showed a strong association of the ERCC3 R109X mutation with colorectal cancer, non-small cell lung cancer (NSCLC), bladder cancer, and glioma. The association of the R109X mutation with colorectal cancer had a p-value of 5.723e-06, a confidence interval of 4.42-26.54, and an odds ratio 11.75. The association of the R109X mutation with NSCLC had a p-value of 0.01972, a confidence interval of 1.06-6.86, and an odds ratio of 2.99. The association of the R109X mutation with bladder cancer had a p-value of 0.004293, a confidence interval of 1.57-12.57, and an odds ratio 5.05. The association of the R109X mutation with glioma had a p-value=0.02462, a confidence interval: 1.00-10.41, and an odds ratio 3.83.

### Example 3

Unless otherwise stated, all materials and methods used in the experiments described in this Example were as for Example 1.

Studies were performed to examine the sensitivity of cells and tumors bearing the ERCC3 R109X mutation to Irofulven. Irofulven (also known as 6-hydroxymethylacylfulvene, HMAF or MGI-114) is an antitumor agent belonging to the Illudin family. Irofulven is a semisynthetic sesquiterpene derivative of Illudin S. Irofulven alkylates DNA and protein macromolecules and arrests cells in the S-phase of the cell cycle.

In vitro experiments were performed to compare the effects of Irofulven on cell viability of ERCC3 WT HMLE cells and CRISPR-edited HMLE cells heterozygous for the ERCC3 R109X mutation. These same cell lines were used in Example 1 to assess sensitivity to IlludinS. Cell viability was assessed 72 hours following treatment of the cells with increasing doses of Irofulven. FIG. 11A provides the results in graphical form. Similar experiments were also performed using cells similar to those described above (i.e. that were either ERCC3 WT or R109X) but that also stably expressed mutant hRAS-V12. (These hRAS-V12 cells were used for xenograft generation in the in vivo experiments described below). FIG. 11B provides results showing the effects of Irofulven on cell viability of the +/+WT H-RAS-V12 HMLE cells the heterozygous+/−R109X H-RAS-V12 HMLE cells. The data shown in both FIG. 11A and FIG. 11B represents the mean of three experiments with error bars representing the SEM. In both cases the ERCC3-mutant (R109X) cells were much more sensitive to Irofulven for a given dose of Irofulven a greater inhibition of cell viability was observed in ERCC3 R109X mutant cells as compared to ERCC3 WT cells.

To assess the effect of the ERCC3 R109X mutation on Irolfulven sensitivity in vivo, a tumor xenograft system was generated and used. As described above, ERCC3 WT and ERCC3-mutant (R109X) cells were made in an HMLE HRAS-V12 background. These cells were injected into the flanks of athymic nude mice. The resultant tumors were allowed to grow to a size of about 100 mm³. Treatment was then started. Either a vehicle control or Irofulven (at a dose of either 3.5 mg/kg or 7 mg/kg) was injected intraperitoneally (IP) daily for 5 consecutive days (the treatment “on” time), followed by a period of 16 days of treatment “off” time—making one 21-day cycle. Treatment cycles were then repeated. This treatment regimen was similar to that employed in several reported human clinical trials of Irofulven. The size of the tumors was measured weekly. FIG. 12 shows the results of such experiments in graphical form. In the ERCC3 WT xenograft group the average tumor size at treatment initiation was 97.7 mm³ (74.6-120.3 mm³). In the ERCC3 R109X xenograft group the average tumor size at treatment initiation was 172.8 mm³ (101.8-238.6 mm³). Irofulven treatment resulted in significant reduction in ERCC3 WT and R109X mutant tumor growth. Furthermore, the data from these preliminary studies also suggested that the ERCC3 R109X mutant tumors may have been more sensitive to these tumor inhibitory effects of Irofulven as compared to the ERCC3 WT tumors. Additional experiments are underway to further validate these preliminary findings.

FIG. 13 shows the results of real time PCR analysis performed on RNA extracted from the cell lines prior to xenograft injections (cell line; black bars), tumors grown from these cell lines following injection into the flanks of athymic nude mice (vehicle; light gray bars), and tumors grown from these cell lines in flanks of athymic nude mice under treatment with Irofulven (Irofulven; dark gray bars) for two treatment cycles (5 consecutive days repeated every 21 days). Data was generated from two independent samples each with 3 technical replicates per experiment. The data showed that ERCC3 expression was upregulated in ERCC3 mutant tumors treated with Irofulven.

### REFERENCE LIST

1. Couch F J, Nathanson K L, Offit K. Two decades after BRCA: setting paradigms in personalized cancer care and prevention. Science. 2014; 343:1466-70.
2. Easton D F, Pharoah P D, Antoniou A C, Tischkowitz M, Tavtigian S V, Nathanson K L, et al. Gene-panel sequencing and the prediction of breast-cancer risk. N Engl J Med. 2015; 372:2243-57.
3. Michailidou K, Beesley J, Lindstrom S, Canisius S, Dennis J, Lush M J, et al. Genome-wide association analysis of more than 120,000 individuals identifies 15 new susceptibility loci for breast cancer. Nat Genet. 2015; 47:373-80.
4. Risinger M A, Groden J. Crosslinks and crosstalk: human cancer syndromes and DNA repair defects. Cancer Cell. 2004; 6:539-45.
5. Brosh R M, Jr. DNA helicases involved in DNA repair and their roles in cancer. Nat Rev Cancer. 2013; 13:542-58.
6. Petrovski S, Wang Q, Heinzen E L, Allen A S, Goldstein D B. Genic intolerance to functional variation and the interpretation of personal genomes. PLoS Genet. 2013; 9:e1003709.
7. Vermeulen W, Scott R J, Rodgers S, Muller H J, Cole J, Arlett C F, et al. Clinical heterogeneity within xeroderma pigmentosum associated with mutations in the DNA repair and transcription gene ERCC3. American journal of human genetics. 1994; 54:191-200.
8. Mcmorris T C, Kelner M J, Wang W, Estes L A, Montoya M A, Taetle R. Structure-Activity-Relationships of Illudins—Analogs with Improved Therapeutic Index. J Org Chem. 1992; 57:6876-83.
9. Marti T M, Hefner E, Feeney L, Natale V, Cleaver J E. H2AX phosphorylation within the G1 phase after UV irradiation depends on nucleotide excision repair and not DNA double-strand breaks. Proceedings of the National Academy of Sciences of the United States of America. 2006; 103:9891-6.
10. Carmi S, Hui K Y, Kochav E, Liu X, Xue J, Grady F, et al. Sequencing an Ashkenazi reference panel supports population-targeted personal genomics and illuminates Jewish and European origins. Nature communications. 2014; 5:4835.
11. Schaeffer L, Roy R, Humbert S, Moncollin V, Vermeulen W, Hoeijmakers J H, et al. DNA repair helicase: a component of BTF2 (TFIIH) basic transcription factor. Science. 1993; 260:58-63.
12. Oh K-S, Khan S G, Jaspers N G J, Raams A, Ueda T, Lehmann A, et al. Phenotypic heterogeneity in the XPB DNA helicase gene (ERCC3): xeroderma pigmentosum without and with Cockayne syndrome. Human mutation. 2006; 27:1092-103.
13. Singh A, Compe E, Le May N, Egly J M. TFIIH subunit alterations causing xeroderma pigmentosum and trichothiodystrophy specifically disturb several steps during transcription. American journal of human genetics. 2015; 96:194-207.
14. Seemanova E. An increased risk for malignant neoplasms in heterozygotes for a syndrome of microcephaly, normal intelligence, growth retardation, remarkable facies, immunodeficiency and chromosomal instability. Mutation research. 1990; 238:321-4.
15. Digweed M, Reis A, Sperling K. Nijmegen breakage syndrome: consequences of defective DNA double strand break repair. BioEssays: news and reviews in molecular, cellular and developmental biology. 1999; 21: 649-56.
16. Bassing C H, Suh H, Ferguson D O, Chua K F, Manis J, Eckersdorff M, et al. Histone H2AX: a dosage-dependent suppressor of oncogenic translocations and tumors. Cell. 2003; 114:359-70.
17. Celeste A, Difilippantonio S, Difilippantonio M J, Fernandez-Capetillo O, Pilch D R, Sedelnikova O A, et al. H2AX haploinsufficiency modifies genomic stability and tumor susceptibility. Cell. 2003; 114:371-83.
18. Goss K H, Risinger M A, Kordich J J, Sanz M M, Straughen J E, Slovek L E, et al. Enhanced tumor formation in mice heterozygous for Blm mutation. Science. 2002; 297:2051-3.
19. Lam M H, Liu Q, Elledge S J, Rosen T M. Chk1 is haploinsufficient for multiple functions critical to tumor suppression. Cancer cell. 2004; 6:45-59.
20. Nikkila J, Parplys A C, Pylkas K, Bose M, Huo Y, Borgmann K, et al. Heterozygous mutations in PALB2 cause DNA replication and damage response defects. Nature communications. 2013; 4:2578.
21. Andressoo J O, Weeda G, de Wit J, Mitchell J R, Beems R B, van Steeg H, et al. An Xpb mouse model for combined xeroderma pigmentosum and cockayne syndrome reveals progeroid features upon further attenuation of DNA repair. Mol Cell Biol. 2009; 29:1276-90.
22. Diderich K, Alanazi M, Hoeijmakers J H. Premature aging and cancer in nucleotide excision repair-disorders. DNA Repair (Amst). 2011; 10:772-80.
23. Itoh T, Cado D, Kamide R, Linn S. DDB2 gene disruption leads to skin tumors and resistance to apoptosis after exposure to ultraviolet light but not a chemical carcinogen. Proceedings of the National Academy of Sciences of the United States of America. 2004; 101:2052-7.
24. Cheo D L, Meira L B, Burns D K, Reis A M, Issac T, Friedberg E C. Ultraviolet B radiation-induced skin cancer in mice defective in the Xpc, Trp53, and Apex (HAP1) genes: genotype-specific effects on cancer predisposition and pathology of tumors. Cancer research. 2000; 60:1580-4.
25. Wijnhoven S W, Kool H J, Mullenders L H, van Zeeland A A, Friedberg E C, van der Horst G T, et al. Age-dependent spontaneous mutagenesis in Xpc mice defective in nucleotide excision repair. Oncogene. 2000; 19:5034-7.
26. Andressoo J O, Mitchell J R, de Wit J, Hoogstraten D, Volker M, Toussaint W, et al. An Xpd mouse model for the combined xeroderma pigmentosum/Cockayne syndrome exhibiting both cancer predisposition and segmental progeria. Cancer cell. 2006; 10:121-32.
27. Shaag A, Walsh T, Renbaum P, Kirchhoff T, Nafa K, Shiovitz S, et al. Functional and genomic approaches reveal an ancient CHEK2 allele associated with breast cancer in the Ashkenazi Jewish population. Human molecular genetics. 2005; 14:555-63.
28. Redston M, Nathanson K L, Yuan Z Q, Neuhausen S L, Satagopan J, Wong N, et al. The APCI1307K allele and breast cancer risk. Nat Genet. 1998; 20:13-4.
29. Weischer M, Bojesen S E, Ellervik C, Tybjaerg-Hansen A, Nordestgaard B G. CHEK2*1100delC genotyping for clinical assessment of breast cancer risk: meta-analyses of 26,000 patient cases and 27,000 controls. J Clin Oncol. 2008; 26:542-8.
30. Alanee S, Couch F, Offit K. Association of a HOXB13 variant with breast cancer. The New England journal of medicine. 2012; 367:480-1.
31. Erkko H, Xia B, Nikkila J, Schleutker J, Syrjakoski K, Mannermaa A, et al. A recurrent mutation in PALB2 in Finnish cancer families. Nature. 2007; 446:316-9.
32. Schnurbein G, Hauke J, Wappenschmidt B, Weber-Lassalle N, Engert S, Hellebrand H, et al. RAD51C deletion screening identifies a recurrent gross deletion in breast cancer and ovarian cancer families. Breast Cancer Res. 2013; 15:R120.
33. Goyal G, Fan T, Silberstein P T. Hereditary cancer syndromes: utilizing DNA repair deficiency as therapeutic target. Fam Cancer. 2016 July; 15(3):359-66.
34. Sonnenblick A, de Azambuja E, Azim H A, Jr., Piccart M. An update on PARP inhibitors—moving to the adjuvant setting. Nat Rev Clin Oncol. 2015; 12:27-41.
35. Chen M C, Zhou B, Zhang K, Yuan Y C, Un F, Hu S, et al. The Novel Ribonucleotide Reductase Inhibitor COH29 Inhibits DNA Repair In Vitro. Mol Pharmacol. 2015; 87:996-1005.
36. Lok B H, Powell S N. Molecular pathways: understanding the role of Rad52 in homologous recombination for therapeutic advancement. Clin Cancer Res. 2012; 18:6400-6.
37. Zhang, F. Genome Engineering Resources—Zhang Lab @ MIT. N.p., 2013. Web. 1 Apr. 2016.
38. Vijai et al. A Recurrent ERCC3 Truncating Mutation Confers Moderate Risk for Breast Cancer; Cancer Discovery 2016; 6:1267-1275.
39. Schilder et al. “A phase 2 evaluation of irofulven as second-line treatment of recurrent or persistent intermediately platinum-sensitive ovarian or primary peritoneal cancer: a Gynecologic Oncology Group trial.” Int J Gynecol Cancer. 2010; 20(7): 1137-1141.
40. Schilder R J, Blessing J A, Pearl M L, et al. Evaluation of irofulven (MGI-114) in the treatment of recurrent or persistent endometrial carcinoma: A phase II study of the Gynecologic Oncology Group. Invest New Drugs. 2004; 22:343-349. [PubMed: 15122083]
41. Sarosy G A, Kotz H, Smith S, et al. Phase II study of irofulven in platinum resistant recurrent ovarian cancer. Proc Am Soc Clin Oncol. 2001; 20 (abstr 868)
42. Seiden M V, Gordon A N, Bodurka D C, et al. A phase II study of irofulven in women with recurrent and heavily pretreated ovarian cancer. Gynecol Oncol. 2006; 101:55-61. [PubMed: 16260029]

We claim:

1. A method of treating cancer, the method comprising administering an effective amount of an Illudin to a subject that has cancer and has a truncating hypomorphic mutation in the ERCC3 gene.

2. The method of claim 1, wherein the subject has a cancer selected from the group consisting of breast cancer, colorectal cancer, NSCLC, bladder cancer, and glioma.

3. The method of claim 1, wherein the subject has a cancer selected from the group consisting of colorectal cancer, NSCLC, bladder cancer, and glioma.

4. The method of claim 1, wherein the subject has breast cancer.

5. The method of claim 1, wherein the cancer is not breast cancer.

6. The method of claim 1, wherein the mutation results in truncation of the protein product of the ERCC3 gene within the first putative helicase domain.

7. The method of claim 6, wherein the subject has a cancer selected from the group consisting of breast cancer, colorectal cancer, NSCLC, bladder cancer, and glioma.

8. The method of claim 6, wherein the subject has a cancer selected from the group consisting of colorectal cancer, NSCLC, bladder cancer, and glioma.

9. The method of claim 6, wherein the subject has breast cancer.

10. The method of claim 6, wherein the cancer is not breast cancer.

11. The method of claim 1, wherein the mutation is an R109X mutation.

12. The method of claim 11, wherein the subject has a cancer selected from the group consisting of breast cancer, colorectal cancer, NSCLC, bladder cancer, and glioma.

13. The method of claim 11, wherein the subject has a cancer selected from the group consisting of colorectal cancer, NSCLC, bladder cancer, and glioma.

14. The method of claim 11, wherein the subject has breast cancer.

15. The method of claim 11, wherein the cancer is not breast cancer.

16. The method of claim 1, wherein the Illudin is selected from the group consisting of: Illudin A, Illudin B, Illudin M, Illudin S, 6-Deoxyilludin M, dehydroilludin M, dihydroilludin M, 6-Deoxyilludin S, dehydroilludin S, dihydroilludin S and Irofulven.

17. The method of claim 6, wherein the Illudin is selected from the group consisting of: Illudin A, Illudin B, Illudin M, Illudin S, 6-Deoxyilludin M, dehydroilludin M, dihydroilludin M, 6-Deoxyilludin S, dehydroilludin S, dihydroilludin S and Irofulven or a derivative of Irofulven.

18. The method of claim 11, wherein the Illudin is selected from the group consisting of: Illudin A, Illudin B, Illudin M, Illudin S, 6-Deoxyilludin M, dehydroilludin M, dihydroilludin M, 6-Deoxyilludin S, dehydroilludin S, dihydroilludin S and Irofulven or a derivative of Irofulven.

19. The method of claim 18, wherein the subject has a cancer selected from the group consisting of breast cancer, colorectal cancer, NSCLC, bladder cancer, and glioma.

20. The method of claim 18, wherein the subject has a cancer selected from the group consisting of colorectal cancer, NSCLC, bladder cancer, and glioma.

21. The method of claim 1, wherein the Illudin is Irofulven.

22. The method of claim 21, wherein the subject has a cancer selected from the group consisting of breast cancer, colorectal cancer, NSCLC, bladder cancer, and glioma.

23. The method of claim 21, wherein the subject has a cancer selected from the group consisting of colorectal cancer, NSCLC, bladder cancer, and glioma.

24. The method of claim 6, wherein the Illudin is Irofulven.

25. The method of claim 24, wherein the subject has a cancer selected from the group consisting of breast cancer, colorectal cancer, NSCLC, bladder cancer, and glioma.

26. The method of claim 24, wherein the subject has a cancer selected from the group consisting of colorectal cancer, NSCLC, bladder cancer, and glioma.

27. The method of claim 11, wherein the Illudin is Irofulven.

28. The method of claim 27, wherein the subject has a cancer selected from the group consisting of breast cancer, colorectal cancer, NSCLC, bladder cancer, and glioma.

29. The method of claim 27, wherein the subject has a cancer selected from the group consisting of colorectal cancer, NSCLC, bladder cancer, and glioma.

30. The method of claim 1, wherein the subject is of Ashkenazi Jewish ancestry.

31. The method of claim 6, wherein the subject is of Ashkenazi Jewish ancestry.

32. The method of claim 11, wherein the subject is of Ashkenazi Jewish ancestry.

33. The method of any of the preceding claims, wherein the subject is of Ashkenazi Jewish ancestry.

34. The method of claim 1, wherein the subject has an estrogen receptor positive (ER+) breast cancer.

35. The method of claim 6, wherein the subject has an estrogen receptor positive (ER+) breast cancer.

36. The method of claim 11, wherein the subject has an estrogen receptor positive (ER+) breast cancer.

37. The method of any of the preceding claims, wherein the subject has an estrogen receptor positive (ER+) breast cancer.

38. The method of claim 1, wherein the subject has a BRCA-negative breast cancer.

39. The method of claim 6, wherein the subject has a BRCA-negative breast cancer.

40. The method of claim 11, wherein the subject has a BRCA-negative breast cancer.

41. The method of any of the preceding claims, wherein the subject has a BRCA-negative breast cancer.

42. The method of claim 1, wherein the effective amount is significantly lower than the amount of the Illudin needed to treat cancer in a subject not having the truncating hypomorphic mutation in the ERCC3 gene.

43. The method of claim 6, wherein the effective amount is significantly lower than the amount of the Illudin needed to treat cancer in a subject not having the truncating hypomorphic mutation in the ERCC3 gene.

44. The method of claim 11, wherein the effective amount is significantly lower than the amount of the Illudin needed to treat cancer in a subject not having the truncating hypomorphic mutation in the ERCC3 gene.

45. The method of any of the preceding claims, wherein the effective amount is significantly lower than the amount of the Illudin needed to treat cancer in a subject not having the truncating hypomorphic mutation in the ERCC3 gene.

46. The method of claim 1, wherein the effective amount is about 70%, or about 60%, or about 50%, or about 40%, or about 30%, or about 20%, or about 10% of the Illudin's maximum tolerated dose.

47. The method of claim 6, wherein the effective amount is about 70%, or about 60%, or about 50%, or about 40%, or about 30%, or about 20%, or about 10% of the Illudin's maximum tolerated dose.

48. The method of claim 11, wherein the effective amount is about 70%, or about 60%, or about 50%, or about 40%, or about 30%, or about 20%, or about 10% of the Illudin's maximum tolerated dose.

49. The method of any of the preceding claims, wherein the effective amount is about 70%, or about 60%, or about 50%, or about 40%, or about 30%, or about 20%, or about 10% of the Illudin's maximum tolerated dose.

50. The method of claim 1, wherein the Illudin is Irofulven, and wherein the effective amount is about is about 70%, or about 60%, or about 50%, or about 40%, or about 30%, or about 20%, or about 10% of the maximum tolerated dose of Irofulven in human subjects.

51. The method of claim 6, wherein the Illudin is Irofulven, and wherein the effective amount is about is about 70%, or about 60%, or about 50%, or about 40%, or about 30%, or about 20%, or about 10% of the maximum tolerated dose of Irofulven in human subjects.

52. The method of claim 11, wherein the Illudin is Irofulven, and wherein the effective amount is about is about 70%, or about 60%, or about 50%, or about 40%, or about 30%, or about 20%, or about 10% of the maximum tolerated dose of Irofulven in human subjects.

53. The method of any of the preceding claims, wherein the Illudin is Irofulven, and wherein the effective amount is about is about 70%, or about 60%, or about 50%, or about 40%, or about 30%, or about 20%, or about 10% of the maximum tolerated dose of Irofulven in human subjects.

54. The method of claim 1, further comprising performing a diagnostic test to determine if the subject has the mutation prior to administering the Illudin to the subject.

55. The method of claim 6, further comprising performing a diagnostic test to determine if the subject has the mutation prior to administering the Illudin to the subject.

56. The method of claim 11, further comprising performing a diagnostic test to determine if the subject has the mutation prior to administering the Illudin to the subject.

57. The method of any of the preceding claims, further comprising performing a diagnostic test to determine if the subject has the mutation prior to administering the Illudin to the subject.

58. The method of any of claims 54-56, wherein the diagnostic test comprises determining the nucleotide sequence of the ERCC3 gene, or of a fragment thereof, in the subject, or in a tissue sample, cell sample, or nucleic acid sample obtained from the subject.

59. The method of claim 58, wherein the diagnostic test comprises determining whether a cytosine (C) or a thymine (T) is present at nucleotide position 325 of a human ERCC3 cDNA, or at a nucleotide position corresponding thereto, in the subject, or in a tissue sample, cell sample, or nucleic acid sample obtained from the subject.

60. The method of claim any of claims 54-59, comprising creating cDNA by reverse transcription.

61. The method of any of claims 54-59, comprising performing PCR or RT PCR.

62. The method of any of claims 54-59, comprising using at least one forward primer comprising SEQ ID NO. 7 or SEQ ID NO. 9 and at least one reverse primer comprising of SEQ ID NO. 8 or SEQ ID NO. 10.

63. The method of any of claims 54-59, comprising contacting a tissue sample, a cell sample, or a nucleic acid sample obtained from the subject with a primer or probe that binds differentially to nucleic acid molecules that comprise the mutation and nucleic acid molecules that do not comprise the mutation.

64. The method of any of claims 54-59, comprising contacting a tissue sample, a cell sample, or a nucleic acid sample obtained from the subject with a mixture or primers or probes, wherein the mixture comprises both (i) a first primer or probe that binds selectively to nucleic acid molecules that comprise the R109X mutation, and (ii) a second primer or probe that binds selectively to nucleic acid molecules that do not comprise the R109X mutation, wherein, optionally, the first and second primers or probes are labeled with different detectable markers.

65. The method of any of claims 54-59, wherein the step of determining the nucleotide sequence comprises performing Sanger sequencing.

66. The method of any of claims 54-59, wherein the step of determining the nucleotide sequence comprises performing Sanger sequencing using a forward primer comprising SEQ ID NO. 1, and a reverse primer comprising SEQ ID NO. 2.

67. The method of any of claims 54-59, comprising performing an allelic discrimination assay.

68. The method of claim 67, comprising using the commercially available C_25963434_10 assay.

69. The method of any of claims 54-59, comprising performing an SNP genotyping assay.

70. The method of any of claims 54-59, comprising using a positive-control tissue sample, cell sample, or nucleic acid sample comprising the ERCC3 mutation.

71. The method of any of claims 54-59, comprising using a negative-control tissue sample, cell sample, or nucleic acid sample not comprising the ERCC3 mutation.

72. The method of any of claims 54-57, wherein the diagnostic test comprises determining whether the mutant ERCC3 protein is present in the subject, or in a tissue sample, cell sample, or protein sample obtained from the subject.

73. The method of claim 72, comprising contacting a tissue sample, a cell sample, or a protein sample obtained from the subject with an anti-ERCC3 antibody.

74. The method of claim 73, wherein the antibody is ARP37963_P050.

75. The method of claim 72, wherein the mutant ERCC3 protein is detected based on its molecular weight, wherein the mutant ERCC3 protein has a molecular weight of about 12 kDa.

76. The method of any of claims 54-57, comprising using a positive-control tissue sample, cell sample, or protein sample comprising the ERCC3 mutation.

77. The method of any of claims 54-57, comprising using a negative-control tissue sample, cell sample, or protein sample not comprising the ERCC3 mutation.

78. A method of determining whether a subject is at risk for developing cancer, the method comprising performing a diagnostic test to determine whether the subject has an R109X mutation in the ERCC3 gene, wherein performing the diagnostic test comprises contacting a tissue sample, cell sample, nucleic acid sample, or protein sample from the subject with a diagnostic reagent selected from the group consisting of:
a. A primer or probe that binds to the ERCC3 gene;
b. A primer or probe that binds differentially to nucleic acid molecules that comprise the R109X mutation and nucleic acid molecules that do not comprise the R109X mutation;
c. A mixture or primers or probes, wherein the mixture comprises both (i) a first primer or probe that binds selectively to nucleic acid molecules that comprise the R109X mutation, and (ii) a second primer or probe that binds selectively to nucleic acid molecules that do not comprise the R109X mutation, wherein, optionally, the first and second primers or probes are labeled with different detectable markers;
d. A primer selected from the group consisting of SEQ ID NO. 1, SEQ ID NO. 2, SEQ ID NO. 7, SEQ ID NO. 8, SEQ ID NO. 9, and SEQ ID NO. 10;
e. An anti-ERCC3 antibody;
f. An antibody that binds differentially to ERCC3 proteins that comprise the R109X mutation that comprise the mutation and nucleic acid molecules that do not comprise the mutation; and
g. A mixture of antibodies, wherein the mixture comprises both (i) a first antibody that binds to proteins that comprise the R109X mutation, and (ii) a second antibody that binds to proteins that do not comprise the R109X mutation, wherein, optionally, the first and second antibodies are labeled with different detectable markers;
wherein, if the R109X mutation is present, the subject at risk for developing cancer.

79. The method of claim 78, wherein the cancer is selected from the group consisting of breast cancer, colorectal cancer, NSCLC, bladder cancer, and glioma.

80. The method of claim 78, wherein the cancer is selected from the group consisting of colorectal cancer, NSCLC, bladder cancer, and glioma.

81. A method of determining whether a subject with cancer is a candidate for treatment with an Illudin, or with a reduced doses of an Illudin, the method comprising performing a diagnostic test to determine whether the subject has an R109X mutation in the ERCC3 gene, wherein performing the diagnostic test comprises contacting a tissue sample, cell sample, nucleic acid sample, or protein sample from the subject with a diagnostic reagent selected from the group consisting of:
a. A primer or probe that binds to the ERCC3 gene;
b. A primer or probe that binds differentially to nucleic acid molecules that comprise the R109X mutation and nucleic acid molecules that do not comprise the R109X mutation;
c. A mixture or primers or probes, wherein the mixture comprises both (i) a first primer or probe that binds selectively to nucleic acid molecules that comprise the R109X mutation, and (ii) a second primer or probe that binds selectively to nucleic acid molecules that do not comprise the R109X mutation, wherein, optionally, the first and second primers or probes are labeled with different detectable markers;
d. A primer selected from the group consisting of SEQ ID NO. 1, SEQ ID NO. 2, SEQ ID NO. 7, SEQ ID NO. 8, SEQ ID NO. 9, and SEQ ID NO. 10;
e. An anti-ERCC3 antibody;
f. An antibody that binds differentially to ERCC3 proteins that comprise the R109X mutation that comprise the mutation and nucleic acid molecules that do not comprise the mutation; and
g. A mixture of antibodies, wherein the mixture comprises both (i) a first antibody that binds to proteins that comprise the R109X mutation, and (ii) a second antibody that binds to proteins that do not comprise the R109X mutation, wherein, optionally, the first and second antibodies are labeled with different detectable markers;
wherein, if the R109X mutation is present, the subject is a candidate for treatment with an Illudin.

82. The method of claim 81, wherein the cancer is selected from the group consisting of breast cancer, colorectal cancer, NSCLC, bladder cancer, and glioma.

83. The method of claim 81, wherein the cancer is selected from the group consisting of colorectal cancer, NSCLC, bladder cancer, and glioma.

84. A method of detecting an R109X mutation in a subject, the method comprising contacting a tissue sample, cell sample, nucleic acid sample, or protein sample from the subject with a diagnostic reagent selected from the group consisting of:
a. A primer or probe that binds to the ERCC3 gene;
b. A primer or probe that binds differentially to nucleic acid molecules that comprise the R109X mutation and nucleic acid molecules that do not comprise the R109X mutation;
c. A mixture or primers or probes, wherein the mixture comprises both (i) a first primer or probe that binds selectively to nucleic acid molecules that comprise the R109X mutation, and (ii) a second primer or probe that binds selectively to nucleic acid molecules that do not comprise the R109X mutation, wherein, optionally, the first and second primers or probes are labeled with different detectable markers;
d. A primer selected from the group consisting of SEQ ID NO. 1, SEQ ID NO. 2, SEQ ID NO. 7, SEQ ID NO. 8, SEQ ID NO. 9, and SEQ ID NO. 10;
e. An anti-ERCC3 antibody;
f. An antibody that binds differentially to ERCC3 proteins that comprise the R109X mutation that comprise the mutation and nucleic acid molecules that do not comprise the mutation; and
g. A mixture of antibodies, wherein the mixture comprises both (i) a first antibody that binds selectively to proteins that comprise the R109X mutation, and (ii) a second antibody that binds selectively to proteins that do not comprise the R109X mutation, wherein, optionally, the first and second antibodies are labeled with different detectable markers.

85. The method of any of claims 78-84, comprising determining the nucleotide sequence of the ERCC3 gene, or of a fragment thereof.

86. The method of any of claims 78-84, comprising determining whether a cytosine (C) or a thymine (T) is present at nucleotide position 325 of a human ERCC3 cDNA, or at a nucleotide position corresponding thereto.

87. The method of any of claims 78-84, comprising creating cDNA by reverse transcription.

88. The method of any of claims 78-84, comprising performing PCR or RT PCR.

89. The method of any of claims 78-84, comprising performing Sanger sequencing.

90. The method of any of claims 78-84, comprising performing an allelic discrimination assay.

91. The method of any of claims 78-84, comprising performing an SNP genotyping assay.

92. The method of any of claims 78-84, wherein the mutant ERCC3 protein is detected based on its molecular weight, wherein the mutant ERCC3 protein has a molecular weight of about 12 kDa.

93. The method of any of claims 78-84, comprising using a positive-control tissue sample, cell sample, nucleic acid sample, or protein sample, comprising the ERCC3 mutation.

94. The method of any of claims 78-84, comprising using a negative-control tissue sample, cell sample, nucleic acid sample, or protein sample, not comprising the ERCC3 mutation.

95. The method of any one of claims 78-84, wherein the subject is of Ashkenazi Jewish ancestry.

96. The method of any one of claims 78-84, wherein the subject has an estrogen receptor positive (ER+) breast cancer.

97. The method of any one of claims 88-84, wherein the subject has a BRCA-negative breast cancer.

98. A diagnostic kit for use in determining whether a subject has an R109X ERCC3 mutation, the kit comprising a diagnostic reagent selected from the group consisting of:
a. A primer or probe that binds to the ERCC3 gene;
b. A primer or probe that binds differentially to nucleic acid molecules that comprise the R109X mutation and nucleic acid molecules that do not comprise the R109X mutation;
c. A mixture or primers or probes, wherein the mixture comprises both (i) a first primer or probe that binds selectively to nucleic acid molecules that comprise the R109X mutation, and (ii) a second primer or probe that binds selectively to nucleic acid molecules that do not comprise the R109X mutation, wherein, optionally, the first and second primers or probes are labeled with different detectable markers;
d. A primer selected from the group consisting of SEQ ID NO. 1, SEQ ID NO. 2, SEQ ID NO. 7, SEQ ID NO. 8, SEQ ID NO. 9, and SEQ ID NO. 10;
e. An anti-ERCC3 antibody;
f. An antibody that binds differentially to ERCC3 proteins that comprise the R109X mutation that comprise the mutation and nucleic acid molecules that do not comprise the mutation; and
g. A mixture of antibodies, wherein the mixture comprises both (i) a first antibody that binds to proteins that comprise the R109X mutation, and (ii) a second antibody that binds to proteins that do not comprise the R109X mutation, wherein, optionally, the first and second antibodies are labeled with different detectable markers;

99. A diagnostic kit according to claim 98, comprising two or more diagnostic reagents (a) through (g).

100. A diagnostic kit according to claim 98, further comprising a positive-control tissue sample, cell sample, nucleic acid sample, or protein sample, comprising the ERCC3 mutation.

101. The diagnostic kit according to claim 98, further comprising a negative-control tissue sample, cell sample, nucleic acid sample, or protein sample, not comprising the ERCC3 mutation.

102. The diagnostic kit according to claim 98, further comprising both a negative-control tissue sample, cell sample, nucleic acid sample, or protein sample, not comprising the ERCC3 mutation, and a positive-control tissue sample, cell sample, nucleic acid sample, or protein sample, comprising the ERCC3 mutation.

103. The diagnostic kit according to claim 98, further comprising instructions for determining whether a subject has an R109X ERCC3 mutation.


SEQUENCE LISTING (17 sequences)

SEQ ID NO: 1 - DNA, 21 bp, Artificial Sequence
  Description of Artificial Sequence Synthetic oligonucleotide
catggagcacctatgcctatt

SEQ ID NO: 2 - DNA, 22 bp, Artificial Sequence
  Description of Artificial Sequence Synthetic oligonucleotide
ctgcaactcatgtttccttgtc

SEQ ID NO: 3 - DNA, 20 bp, Artificial Sequence
  Description of Artificial Sequence Synthetic oligonucleotide
tgtggtgttgggcagcttat

SEQ ID NO: 4 - DNA, 20 bp, Artificial Sequence
  Description of Artificial Sequence Synthetic oligonucleotide
acactcactttgggctgcat

SEQ ID NO: 5 - DNA, 19 bp, Artificial Sequence
  Description of Artificial Sequence Synthetic oligonucleotide
catctccttctcggcatca

SEQ ID NO: 6 - DNA, 20 bp, Artificial Sequence
  Description of Artificial Sequence Synthetic oligonucleotide
aaccctgttgtcaatgcctc

SEQ ID NO: 7 - DNA, 22 bp, Artificial Sequence
  Description of Artificial Sequence Synthetic oligonucleotide
gtccgcgaagatgacaaaattg

SEQ ID NO: 8 - DNA, 21 bp, Artificial Sequence
  Description of Artificial Sequence Synthetic oligonucleotide
aattcaggagacatagggcac

SEQ ID NO: 9 - DNA, 19 bp, Artificial Sequence
  Description of Artificial Sequence Synthetic oligonucleotide
atgggcaaaagagaccgag

SEQ ID NO: 10 - DNA, 20 bp, Artificial Sequence
  Description of Artificial Sequence Synthetic oligonucleotide
ctgactcatccacctgcttc

SEQ ID NO: 11 - protein, 782 aa, Homo sapiens
MGKRDRADRDKKKSRKRHYEDEEDDEEDAPGNDPQEAVPSAAGKQVDESGTKVDEYGAKDYRLQMPLKDDHTSRPLWVAPDGHIFLEAFSPVYKYAQDFLVAIAEPVCRPTHVHEYKLTAYSLYAAVSVGLQTSDITEYLRKLSKTGVPDGIMQFIKLCTVSYGKVKLVLKHNRYFVESCHPDVIQHLLQDPVIRECRLRNSEGEATELITETFTSKSAISKTAESSGGPSTSRVTDPQGKSDIPMDLFDFYEQMDKDEEEEEETQTVSFEVKQEMIEELQKRCIHLEYPLLAEYDFRNDSVNPDINIDLKPTAVLRPYQEKSLRKMFGNGRARSGVIVLPCGAGKSLVGVTAACTVRKRCLVLGNSAVSVEQWKAQFKMWSTIDDSQICRFTSDAKDKPIGCSVAISTYSMLGHTTKRSWEAERVMEWLKTQEWGLMILDEVHTIPAKMFRRVLTIVQAHCKLGLTATLVREDDKIVDLNFLIGPKLYEANWMELQNNGYIAKVQCAEVWCPMSPEFYREYVAIKTKKRILLYTMNPNKFRACQFLIKFHERRNDKIIVFADNVFALKEYAIRLNKPYIYGPTSQGERMQILQNFKHNPKINTIFISKVGDTSFDLPEANVLIQISSHGGSRRQEAQRLGRVLRAKKGMVAEEYNAFFYSLVSQDTQEMAYSTKRQRFLVDQGYSFKVITKLAGMEEEDLAFSTKEEQQQLLQKVLAATDLDAEEEVVAGEFGSRSSQASRRFGTMSSMSGADDTVYMEYHSSRSKAPSKHVHPLFKRFRK

SEQ ID NO: 12 - DNA, 2751 bp, Homo sapiens
gggagcttccggattgagccggaagtccccccagagcggatgccgcggcgggcctgtgggagcggggtcatcttctctctgctgctgtagctgccatgggcaaaagagaccgagcggaccgcgacaagaagaaatccaggaagcggcactatgaggatgaagaggatgatgaagaggacgccccggggaacgaccctcaggaagcggttccctcggcggcggggaagcaggtggatgagtcaggcaccaaagtggatgaatatggagccaaggactacaggctgcaaatgccgctgaaggacgaccacacctccaggcccctctgggtggctcccgatggccatatcttcttggaagccttctctccagtttacaaatatgcccaagacttcttggtggctattgcagagccagtgtgccgaccaacccatgtgcatgagtacaaactaactgcctactccttgtatgcagctgtcagcgttgggctgcaaaccagtgacatcaccgagtacctcaggaagctcagcaagactggagtccctgatggaattatgcagtttattaagttgtgtactgtcagctatggaaaagtcaagctggtcttgaagcacaacagatacttcgttgaaagttgccaccctgatgtaatccagcatcttctccaggaccccgtgatccgagaatgccgcttaagaaactctgaaggggaggccactgagctcatcacagagactttcacaagcaaatctgccatttctaagactgctgaaagcagtggtgggccctccacttcccgagtgacagatccacagggtaaatctgacatccccatggacctgtttgacttctatgagcaaatggacaaggatgaagaagaagaagaagagacacagacagtgtcttttgaagtcaagcaggaaatgattgaggaactccagaaacgttgcatccacctggagtaccctctgttggcagaatatgacttccggaatgattctgtcaaccctgatatcaacattgacctaaagcccacagctgtcctcagaccctatcaggagaagagcttgcgaaagatgtttggaaacgggcgtgcacgttcgggggtcattgttcttccctgcggtgctggaaagtccctggttggtgtgactgctgcatgcactgtcagaaaacgctgtctggtgctgggcaactcagctgtttctgtggagcagtggaaagcccagttcaagatgtggtccaccattgacgacagccagatctgccggttcacctccgatgccaaggacaagcccatcggctgctccgttgccattagcacctactccatgctgggccacaccaccaaaaggtcctgggaggccgagcgagtcatggagtggctcaagacccaggagtggggcctcatgatcctggatgaagtgcacaccataccagccaagatgttccgaagggtgctcaccatcgtgcaggcccactgtaagctgggtttgactgcgaccctcgtccgcgaagatgacaaaattgtggatttaaattttctgattgggcctaagctctacgaagccaactggatggagctgcagaataatggctacatcgccaaagtccagtgtgctgaggtctggtgccctatgtctcctgaattttaccgggaatatgtggcaatcaaaaccaagaaacgaatcttgctgtacaccatgaaccccaacaaatttagagcttgccagtttctgatcaagtttcatgaaaggaggaatgacaagattattgtctttgctgacaatgtgtttgccctaaaggaatatgccattcgactgaacaaaccctatatctacggacctacgtctcagggggaaaggatgcaaattctccagaatttcaagcacaaccccaaaattaacaccatcttcatatccaaggtaggtgacacttcgtttgatctgccggaagcaaatgtcctcattcagatctcatcccatggtggctccaggcgtcaggaagcccaaaggctagggcgggtgcttcgagctaaaaaagggatggttgcagaagagtacaatgcctttttctactcactggtatcccaggacacacaggaaatggcttactcaaccaagcggcagagattcttggtagatcaaggttatagcttcaaggtgatcacgaaactcgctggcatggaggaggaagacttggcgttttcgacaaaagaagagcaacagcagctcttacagaaagtcctggcagccactgacctggatgccgaggaggaggtggtggctggggaatttggctccagatccagccaggcatctcggcgctttggcaccatgagttctatgtctggggccgacgacactgtgtacatggagtaccactcatcgcggagcaaggcgcccagcaaacatgtacacccgctcttcaagcgctttaggaaatgatgcttaggcagggtacttcgttcaagaccggcgcttggcacccttgttggaaagggattttcagcataacattttccttccacctctttgaccttccctccagcgttggccaaattgtgctgaggaagatgcatcaagggcttggctgtgccttcataggtcatctagggttttataaaggaggaggagacaatattttttcaaactttttggggagtggggtcatttctgtatataaaaaatgttaatatttaaggtgtatttatgttaccgttctgaataaacagaatggaccattgaaccagta

SEQ ID NO: 13 - DNA, 2349 bp, Homo sapiens
atgggcaaaagagaccgagcggaccgcgacaagaagaaatccaggaagcggcactatgaggatgaagaggatgatgaagaggacgccccggggaacgaccctcaggaagcggttccctcggcggcggggaagcaggtggatgagtcaggcaccaaagtggatgaatatggagccaaggactacaggctgcaaatgccgctgaaggacgaccacacctccaggcccctctgggtggctcccgatggccatatcttcttggaagccttctctccagtttacaaatatgcccaagacttcttggtggctattgcagagccagtgtgccgaccaacccatgtgcatgagtacaaactaactgcctactccttgtatgcagctgtcagcgttgggctgcaaaccagtgacatcaccgagtacctcaggaagctcagcaagactggagtccctgatggaattatgcagtttattaagttgtgtactgtcagctatggaaaagtcaagctggtcttgaagcacaacagatacttcgttgaaagttgccaccctgatgtaatccagcatcttctccaggaccccgtgatccgagaatgccgcttaagaaactctgaaggggaggccactgagctcatcacagagactttcacaagcaaatctgccatttctaagactgctgaaagcagtggtgggccctccacttcccgagtgacagatccacagggtaaatctgacatccccatggacctgtttgacttctatgagcaaatggacaaggatgaagaagaagaagaagagacacagacagtgtcttttgaagtcaagcaggaaatgattgaggaactccagaaacgttgcatccacctggagtaccctctgttggcagaatatgacttccggaatgattctgtcaaccctgatatcaacattgacctaaagcccacagctgtcctcagaccctatcaggagaagagcttgcgaaagatgtttggaaacgggcgtgcacgttcgggggtcattgttcttccctgcggtgctggaaagtccctggttggtgtgactgctgcatgcactgtcagaaaacgctgtctggtgctgggcaactcagctgtttctgtggagcagtggaaagcccagttcaagatgtggtccaccattgacgacagccagatctgccggttcacctccgatgccaaggacaagcccatcggctgctccgttgccattagcacctactccatgctgggccacaccaccaaaaggtcctgggaggccgagcgagtcatggagtggctcaagacccaggagtggggcctcatgatcctggatgaagtgcacaccataccagccaagatgttccgaagggtgctcaccatcgtgcaggcccactgtaagctgggtttgactgcgaccctcgtccgcgaagatgacaaaattgtggatttaaattttctgattgggcctaagctctacgaagccaactggatggagctgcagaataatggctacatcgccaaagtccagtgtgctgaggtctggtgccctatgtctcctgaattttaccgggaatatgtggcaatcaaaaccaagaaacgaatcttgctgtacaccatgaaccccaacaaatttagagcttgccagtttctgatcaagtttcatgaaaggaggaatgacaagattattgtctttgctgacaatgtgtttgccctaaaggaatatgccattcgactgaacaaaccctatatctacggacctacgtctcagggggaaaggatgcaaattctccagaatttcaagcacaaccccaaaattaacaccatcttcatatccaaggtaggtgacacttcgtttgatctgccggaagcaaatgtcctcattcagatctcatcccatggtggctccaggcgtcaggaagcccaaaggctagggcgggtgcttcgagctaaaaaagggatggttgcagaagagtacaatgcctttttctactcactggtatcccaggacacacaggaaatggcttactcaaccaagcggcagagattcttggtagatcaaggttatagcttcaaggtgatcacgaaactcgctggcatggaggaggaagacttggcgttttcgacaaaagaagagcaacagcagctcttacagaaagtcctggcagccactgacctggatgccgaggaggaggtggtggctggggaatttggctccagatccagccaggcatctcggcgctttggcaccatgagttctatgtctggggccgacgacactgtgtacatggagtaccactcatcgcggagcaaggcgcccagcaaacatgtacacccgctcttcaagcgctttaggaaatga

SEQ ID NO: 14 - DNA, 21 bp, Homo sapiens
  modified_base (11)..(11): a, c, t, g, unknown or other
  modified_base (15)..(15): a, c, t, g, unknown or other
  modified_base (19)..(19): a, c, t, g, unknown or other
  modified_base (21)..(21): a, c, t, g, unknown or other
ttgcagagccnatgngccnan

SEQ ID NO: 15 - DNA, 21 bp, Homo sapiens
agagccagtgagccaaccaag

SEQ ID NO: 16 - DNA, 21 bp, Homo sapiens
  modified_base (11)..(11): a, c, t, g, unknown or other
  modified_base (14)..(15): a, c, t, g, unknown or other
  modified_base (18)..(21): a, c, t, g, unknown or other
tgccgaccaanccnnggnnnn

SEQ ID NO: 17 - DNA, 21 bp, Homo sapiens
  modified_base (11)..(11): a, c, t, g, unknown or other
gccagtgtgcngaccaaccca